(19) United States
(12) Patent Application Publication
(10) Pub. No.: US 2026/0345836 A1
(43) Pub. Date: Nov. 5, 2026

(54) IN VIVO DETECTION OF IMMUNE CELLS

(71) Applicant: Biosceptre (Aust) Pty Ltd, North Ryde, New South Wales (AU)

(72) Inventor: Patrick Schlegel, North Ryde, New South Wales (AU)

(21) Appl. No.: 19/110,326

(22) PCT Filed: Sep. 14, 2023
(86) PCT No.: PCT/AU2023/050888

§ 371 Date: Mar. 10, 2025

(30) Foreign Application Priority Data
Sep. 14, 2022 (AU) 2022902654

(51) Int. Cl. A61K 51/08; C07K 14/435; G01N 33/534

(52) U.S. Cl. CPC A61K 51/088; C07K 14/7916; G01N 33/534; A61K 2123/00; C07K 2319/30; G01N 2333/705

(57) ABSTRACT

The present invention relates to radiolabelled molecules, and related precursor molecules, for use in detecting immune cells expressing a receptor comprising an antigen binding site for binding to dysfunctional P2X7 receptor and a signalling domain.

### FIELD OF THE INVENTION

The present invention relates to radiolabelled compounds for detecting immune cells, radiolabel-precursor compounds for preparing radiolabelled compounds, and uses and methods thereof.

### RELATED APPLICATION

This application claims priority from Australian provisional application AU 2022902654, the entire contents of which are hereby incorporated by reference.

### BACKGROUND OF THE INVENTION

Cancer immunotherapy is a rapidly growing field. The development of T cells expressing chimeric antigen receptors (CARs) has revolutionised adoptive cell therapies.

The potential of this approach has been demonstrated in clinical trials, wherein CAR T cells were infused into adult and paediatric patients with B-cell malignancies, neuroblastoma, and sarcoma. To date, over 500 clinical trials have emerged worldwide, designed at testing the efficacy of CAR T cells targeted to bind 64 different tumour associated antigens. Among these, three CD19-specific CAR T cell products have been approved for the treatment of acute lymphoblastic leukaemia (ALL), large B cell lymphoma and mantle cell lymphoma. To date, most of the success with CAR T therapies has been observed in the context of so-called “liquid” tumours, or where the CARs are directed to CD19, CD22 or the B cell maturation antigen (BCMA).

Several challenges remain in the clinical application of CAR T cell therapies. In view of the challenges associated with CAR T cell therapies, there is interest in developing approaches for imaging and tracking CAR T cells in vivo to gain further insight into their biological functions. These approaches may also be useful for monitoring and adjusting treatments involving CAR T cell therapies.

Current methods for monitoring infused CAR T cells include serum profiling of cytokines associated with T cell activation, direct enumeration of tumour-specific T cells in peripheral blood, and (repeated) tumour biopsies. However, these methods do not allow for real time or quantitative monitoring of infused CAR T cells in vivo. Labelled antibodies for tracking T cells have also been developed. However, a drawback associated with this method is that the antigen expressed on the target cell is typically also expressed to some extent in other tissue, which may result in unspecific background uptake of the labelled antibodies.

Therefore, there is a need for alternative approaches for imaging and tracking immune cells such as CAR T cells in vivo.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention provides a radiolabelled molecule comprising:
(i) a dysfunctional P2X₇ receptor epitope moiety that is recognised or capable of being bound by an antigen recognition domain of a receptor expressed on an immune cell, wherein the receptor is for binding a dysfunctional P2X₇ receptor and comprises a signalling domain; and
(ii) a radionuclide that is directly or indirectly linked to the epitope moiety,
or a salt or solvate thereof.

The present invention provides a radiolabelled molecule comprising:
(i) a peptide and
(ii) a radionuclide that is directly or indirectly linked to the peptide,
or a salt or solvate thereof,
wherein the peptide comprises or consists of the amino acid sequence of a linear epitope derived from the P2X₇ receptor, preferably the amino acid sequence of SEQ ID NO: 14 or SEQ ID NO: 7. Optionally the peptide comprises or consists of the amino acid sequence GHNYTTRNILPGLNITC (SEQ ID NO: 2; also referred to herein as the “E200 epitope”); GHNYTTRNILPGLNIT (SEQ ID NO: 3); KYYKENNVEKRTLIK (SEQ ID NO: 4; also referred to herein as the “E300” epitope); or GHNYTTRNILPGAGAKYYKENNVEK (SEQ ID NO: 6; also referred to herein as the “E200/E300” or “composite” epitope), or any of SEQ ID NOs: 2 to 69 and 122.

Preferably the peptide is recognised or capable of being bound by an antigen recognition domain of an exogenous cell surface receptor comprising an intracellular signalling domain (eg chimeric antigen receptors, including expressed on T cells).

Preferably, the radionuclide may be a beta-emitting radioisotope (such as a positron or beta plus decay) or a gamma-emitting radioisotope.

In any embodiment, the radionuclide is selected from: ¹¹C, ¹⁸F, ⁴⁴Sc, ⁶²Cu, ⁶⁴Cu, ⁶⁷Cu ⁶⁷Ga, ⁶⁸Ga, ⁸⁶Y, ⁹⁰Y, ⁸⁹Zr, ⁹⁰Nb, ^{94m}Tc, ⁹⁹mTc, ¹¹¹In, ¹²³I, ¹²⁴I, ¹²⁵I, ¹³¹I, ¹⁷⁷Lu and ²¹³Bi.

The radionuclide may be directly linked to the epitope moiety, for example directly linked to an amino acid side chain of the epitope moiety. Alternatively, the radionuclide may be indirectly linked to the epitope moiety, for example the radionuclide may be contained in a radiolabelled moiety that is conjugated to the epitope moiety. In some embodiments, the radiolabelled moiety comprises a covalently bound radionuclide.

In other embodiments, the radiolabelled moiety comprises a chelator moiety that is capable of chelating a radionuclide, wherein a radionuclide is complexed with the chelator moiety. The chelator moiety may be selected from TMT, DOTA, TCMC, DO3A, CB-DO2A, NOTA, NETA, diamsar, DTPA, CHX-A″-DTPA, TETA, 11-tetraacetic acid, Te2A, HBED, 5HBED, HYBIC, DFO, DFOsq and HOPO.

In any embodiment, the radiolabelled moiety is conjugated to a further peptide (eg dysfunctional P2X₇ receptor epitope moiety) that is recognised or capable of being bound by an antigen recognition domain of a receptor expressed on an immune cell, preferably wherein the receptor is for binding a dysfunctional P2X₇ receptor and comprises a signalling domain.

In a further aspect, the present invention provides a radiolabel-precursor molecule comprising:
(i) a dysfunctional P2X₇ receptor epitope moiety that is recognised or capable of being bound by an antigen recognition domain of a receptor expressed on an immune cell, wherein the antigen recognition domain is for binding a dysfunctional P2X₇ receptor and the receptor comprises a signalling domain; and
(ii) one of:
   (A) an atom or functionality capable of being converted to a radionuclide;
   (B) a reactive functionality capable of conjugating to a radiolabelled prosthetic group; or
   (C) a chelator moiety capable of chelating a radionuclide, or a salt or solvate thereof.

In any embodiment, the atom or functionality may be any suitable atom or functionality capable of being converted to a radionuclide. The atom or functionality may be converted to a radionuclide, for example, by substitution, addition or exchange with a compound comprising the radionuclide. In some embodiments, the atom or functionality is capable of being substituted with ¹⁸F. In some embodiments, the atom or functionality is capable of undergoing isotopic exchange with ¹²⁵I.

In any embodiment, the reactive functionality may be any suitable reactive functionality capable of conjugating a radiolabelled prosthetic group. It will be appreciated that the radiolabelled prosthetic group comprises a radionuclide, which may be linked to the radiolabelled prosthetic group by a covalent bond or a non-covalent bond (eg by co-ordination). In certain embodiments, the reactive functionality is capable of reacting with a radiolabelled prosthetic group via click chemistry.

In any embodiment, the chelator moiety may be any suitable chelator moiety capable of chelating a radionuclide. In certain embodiments, the chelator moiety may be selected from TMT, DOTA, TCMC, DO3A, CB-DO2A, NOTA, NETA, diamsar, DTPA, CHX-A″-DTPA, TETA, 11-tetraacetic acid, Te2A, HBED, 5HBED, HYBIC, DFO, DFOsq and HOPO.

In certain embodiments, the radiolabelled prosthetic group may be conjugated (or capable of being further conjugated) to a further dysfunctional P2X₇ receptor epitope moiety that is recognised or capable of being bound by an antigen recognition domain of a receptor expressed on an immune cell, wherein the receptor is for binding a dysfunctional P2X₇ receptor and comprises a signalling domain.

In a further aspect, the present invention provides a method of preparing a radiolabelled molecule, comprising:
providing a radiolabel-precursor molecule as defined herein; and
reacting the radiolabel-precursor molecule under conditions suitable for:
(i) converting the atom or functionality of (A) to a radionuclide;
(ii) conjugating the reactive functionality of (B) to a radiolabelled prosthetic group; or
(ii) chelating the chelator moiety of (C) to a radionuclide.
thereby providing a radiolabelled molecule.

In a further aspect, the present invention provides the use of a radiolabelled molecule described herein for detecting an immune cell expressing a receptor comprising an antigen recognition domain for binding a dysfunctional P2X₇ receptor and a signalling domain.

In another aspect, the present invention provides a method of detecting an immune cell expressing a receptor comprising an antigen recognition domain for binding a dysfunctional P2X₇ receptor and a signalling domain, in a subject, comprising:
administering a radiolabelled molecule described herein to a subject who has been administered an immune cell expressing a receptor comprising an antigen recognition domain for binding a dysfunctional P2X₇ receptor and a signalling domain; and
detecting the radiolabelled molecule in the subject.

In any embodiment of this aspect, the radiolabelled molecule is detected by performing a radionuclide scan. The radionuclide scan may be a positron emission tomography (PET) scan or a single-photon emission computerized tomography (SPECT) scan.

In some embodiments of this aspect, the method further comprises allowing the radiolabelled molecule to concentrate at sites in the subject where the immune cell is found, prior to the step of detecting the radiolabelled molecule.

In some embodiments of this aspect, the method further comprises administering an immune cell expressing a receptor comprising an antigen recognition domain for binding a dysfunctional P2X₇ receptor and a signalling domain, to the subject, prior to the step of administering the radiolabelled molecule.

In another aspect, the present invention provides a composition comprising a radiolabelled molecule described herein, or a salt or solvent thereof, or the radiolabel-precursor molecule described herein, or a salt or solvate thereof.

The present invention provides a radiolabel-precursor molecule comprising:
(i) a peptide; and
(ii) one of:
   (A) an atom or functionality capable of being converted to a radionuclide;
   (B) a reactive functionality capable of conjugating to a radiolabelled prosthetic group; or
   (C) a chelator moiety capable of chelating a radionuclide,
or a salt or solvate thereof,
wherein the peptide comprises or consists of the amino acid sequence of SEQ ID NO: 14 (preferably wherein the peptide comprises or consists of the amino acid sequence of SEQ ID NO: 7). Optionally the peptide comprises or consists of the amino acid sequence GHNYTTRNILPGLNITC (SEQ ID NO: 2; also referred to herein as the “E200 epitope”); GHNYTTRNILPGLNIT (SEQ ID NO: 3); KYYKENNVEKRTLIK (SEQ ID NO: 4; also referred to herein as the “E300” epitope); or GHNYTTRNILPGAGAKYYKENNVEK (SEQ ID NO: 6; also referred to herein as the “E200/E300” or “composite” epitope) or any of SEQ ID NOs: 2 to 69 and 122.

Preferably the peptide is recognised or capable of being bound by an antigen recognition domain of an exogenous cell surface receptor comprising an intracellular signalling domain (eg chimeric antigen receptors, including expressed on T cells).

The present invention provides a method of detecting an immune cell expressing an exogenous cell surface receptor comprising an intracellular signalling domain (eg a chimeric antigen receptor, including expressed on T cells), wherein the receptor comprises an antigen recognition domain for binding to a peptide comprising or consisting of the amino acid sequence of SEQ ID NO: 14 (preferably comprising or consisting of the amino acid sequence of SEQ ID NO: 7);
administering a radiolabelled molecule described herein to a subject who has been administered an immune cell expressing the receptor;
detecting the radiolabelled molecule in the subject.

Preferably, the radiolabelled molecule is detected by performing a radionuclide scan. The radionuclide scan may be a positron emission tomography (PET) scan or a single-photon emission computerized tomography (SPECT) scan.

Optionally, the antigen recognition domain of the receptor is for binding to a peptide comprising or consisting of the amino acid sequence GHNYTTRNILPGLNITC (SEQ ID NO: 2; also referred to herein as the “E200 epitope”); GHNYTTRNILPGLNIT (SEQ ID NO: 3); KYYKENNVEKRTLIK (SEQ ID NO: 4; also referred to herein as the “E300” epitope); or GHNYTTRNILPGAGAKYYKENNVEK (SEQ ID NO: 6; also referred to herein as the “E200/E300” or “composite” epitope), or any of SEQ ID NOs: 2 to 69 and 122.

In any aspect of the invention, the dysfunctional P2X₇ receptor epitope moiety may be provided in the form of a dysfunctional P2X₇ receptor, or a fragment of a dysfunctional P2X₇ receptor that has at least one of the three ATP binding sites that are formed at the interface between adjacent correctly packed monomers that are unable to bind ATP. Such receptors are generally understood to be unable to extend the opening of the non-selective calcium channels to apoptotic pores.

In any aspect, the dysfunctional P2X₇ receptor epitope moiety comprises or consists of a fragment of a dysfunctional P2X₇ receptor. Exemplary fragments include peptides comprising the amino acid sequence GHNYTTRNILPGLNITC (SEQ ID NO: 2; also referred to herein as the “E200 epitope”); GHNYTTRNILPGLNIT (SEQ ID NO: 3); KYYKENNVEKRTLIK (SEQ ID NO: 4; also referred to herein as the “E300” epitope); or GHNYTTRNILPGAGAKYYKENNVEK (SEQ ID NO: 6; also referred to herein as the “E200/E300” or “composite” epitope). Other exemplary peptide fragments are included in Table 1 herein and include any of SEQ ID NOs: 2 to 69 and 122. Preferably the dysfunctional P2X₇ receptor epitope moiety comprises at least the sequence of SEQ ID NO: 7.

The dysfunctional P2X₇ receptor epitope moiety may additionally comprise a spacer region linking the moiety to the radiolabel. Examples of such spacer sequences are further provided herein.

In preferred embodiments of any aspect of the invention, the architecture and spatial arrangement of a radiolabelled molecule, or radiolabel precursor molecule of the invention, is such that the minimum sequence of SEQ ID NO: 14, more preferably SEQ ID NO: 7, is available for binding to an antigen binding domain of a receptor expressed on an immune cell, as described herein (e.g., a chimeric antigen receptor for binding dysfunctional P2X₇ receptor). In other words, the architecture is such that there is no steric hindrance which would prevent binding of the antigen binding domain to the peptide.

In preferred embodiments, the architecture of a radiolabelled molecule or radiolabel precursor molecule of the invention provides a radionuclide that is directly or indirectly linked to the epitope moiety via the C-terminal region of the epitope moiety.

Alternatively, the architecture of a radiolabelled molecule or radiolabel precursor molecule of the invention provides a radionuclide that is directly or indirectly linked to the epitope moiety via the N-terminal region of the epitope moiety.

In preferred embodiments, the architecture of a radiolabel precursor molecule of the invention provides an atom or functionality capable of being converted to a radionuclide that is directly or indirectly linked to the epitope moiety via the C-terminal region of the epitope moiety.

In further embodiments, the architecture of a radiolabel precursor molecule of the invention provides a reactive functionality capable of conjugating to a radiolabelled prosthetic group that is directly or indirectly linked to the epitope moiety via the C-terminal region of the epitope moiety.

In further embodiments, the architecture of a radiolabel precursor molecule of the invention provides a chelator moiety capable of chelating a radionuclide that is directly or indirectly linked to the epitope moiety via the C-terminal region of the epitope moiety.

Alternatively, the architecture of a radiolabel precursor molecule of the invention provides: (A) an atom or functionality capable of being converted to a radionuclide; (B) a reactive functionality capable of conjugating to a radiolabelled prosthetic group; or (C) a chelator moiety capable of chelating a radionuclide, that is directly or indirectly linked to the epitope moiety via the N-terminal region of the epitope moiety.

In accordance with any aspect of the invention, the radiolabelled molecule or radiolabel precursor molecule may be in the form of a fusion protein. Optionally the fusion protein comprises the peptide (eg the linear epitope of a P2X₇ receptor, such as in SEQ ID NO: 7 or 14, or as defined in any of SEQ ID NOs: 2 to 69 and 122).joined to an Fc region of an antibody, as further herein described. Accordingly, the present invention provides a fusion protein comprising a peptide derived from the P2X₇ receptor (eg dysfunctional P2X₇ receptor epitope moiety) and an Fc region of an antibody, optionally wherein the fusion protein comprises a radiolabel or a moiety capable of being radiolabelled, or wherein the fusion protein comprises one of:
(A) an atom or functionality capable of being converted to a radionuclide;
(B) a reactive functionality capable of conjugating to a radiolabelled prosthetic group; or
(C) a chelator moiety capable of chelating a radionuclide, or a salt or solvate thereof.

Examples of such fusion proteins are provided herein at SEQ ID NOs: 145 to 158, 160 and 161

In preferred embodiments, the fusion protein comprising a dysfunctional P2X₇ receptor epitope moiety and an Fc region of an antibody, preferably comprises one or more modifications to the Fc region, for example, to reduce effector function (through attenuating or reducing capacity to bind to Fc receptors and/or by reducing or ablating recruitment of complement C1q), to reduce serum half-life (through attenuating or reducing capacity to bind to the FcRN receptor), and/or by reducing the propensity of the Fc region to aggregate and dimerise. Relevant amino acid substitutions for altering effector function, serum half-life and aggregation are well known to the skilled person and further described herein, including as exemplified in Table 1.

The present invention also provides a heterodimeric asymmetric molecule comprising a fusion protein as described herein (eg comprising a peptide of SEQ ID NO: 7 or 14, or variations thereof as exemplified in any of SEQ ID NOs: 2 to 69) and an Fc region of an antibody, and further comprising an Fc region of an antibody that does not comprise the peptide. Such asymmetric heterodimeric molecules may be obtained using the knob-in-hole technology, as further described herein, for facilitating dimerisation of non-identical Fc regions.

Preferably, the fusion protein or the heterodimeric asymmetric molecule, consists or consists essentially of the peptide and an Fc region of an antibody, such that the fusion protein or heterodimeric asymmetric molecule does not comprise an antigen binding domain of an antibody (ie such that the fusion protein does not comprise a VH, VL, Fab, Fv, or an scFv derived from an antibody).

It will be appreciated that the radiolabel, the atom or functionality capable of being converted to a radionuclide, the reactive functionality capable of conjugating to a radiolabelled prosthetic group, or the chelator moiety may be linked to or a part of the dysfunctional P2X₇ receptor epitope moiety of the fusion protein, or may be linked to or a part of the Fc region of the fusion protein.

In any aspect, the dysfunctional P2X₇ receptor epitope moiety is bound or capable of being bound by an antigen binding protein, or antigen binding fragment thereof, that binds to dysfunctional P2X₇ receptors, but is not bound or is not capable of being bound by an antigen binding protein, or antigen binding fragment thereof that binds to functional P2X₇ receptors. Examples of suitable antigen binding proteins, or fragments thereof are further described herein.

The radiolabelled molecule or radiolabel-precursor molecule may comprise 2 or more peptides as described herein (eg 2 or more dysfunctional P2X₇ receptor epitope moieties). The 2 or more peptides may comprise or consist of the same sequence, or of different sequences. For example, in any aspect, a radiolabelled molecule or radiolabel-precursor molecule may comprise a peptide in the form of the E200 epitope and a further peptide in the form of the E300 epitope. Alternatively, in any aspect, a radiolabelled molecule may comprise a peptide in the form of the E200 epitope and a further peptide in the form of the composite epitope. Still further, in any aspect, a radiolabelled molecule, or radiolabel-precursor molecule may comprise a first peptide in the form of the E200 epitope and a further peptide in the form of the E200 epitope.

In any aspect of the invention, the receptor comprising the antigen recognition domain and signalling domain, may be a chimeric antigen receptor (CAR), or variant thereof including a ligand-based CAR, or modified T cell receptor (TCR) or the like.

In any aspect of the invention, the immune cell may be a leukocyte, a Peripheral Blood Mononuclear Cell (PBMC), a lymphocyte, a T cell, a CD4+ T cell, a CD8+ T cell, a natural killer cell, a natural killer T cell, or a γδ T cell. In any embodiment, the cell may be a T cell, wherein optionally said T cell does not express TcRαβ, PD1, CD3 or CD96 (e.g. by way of knocking down or knocking out one of these genes on a genetic level or functional level). In any embodiment, the cell may be an immune cell, wherein optionally said cell does not express accessory molecules that can be checkpoint, exhaustion or apoptosis-associated signalling receptors as well as ligands such as PD-1, LAG-3, TIGIT, CTLA-4, FAS-L and FAS-R, (e.g. by way of knocking out one of these genes on a genetic level or functional level).

In preferred embodiments of any aspect of the invention, the immune cell is a T cell or other effector cell expressing a CAR comprising an antigen binding domain for binding dysfunctional P2X₇ receptor.

In another aspect, the present invention provides a formulation comprising a radiolabelled molecule described herein, or a salt or solvent thereof, or a radiolabel-precursor molecule described herein, or a salt or solvate thereof.

In another aspect, the present invention provides a kit comprising one or more of the following:
(i) a radiolabelled molecule described herein, or a salt or solvate thereof;
(ii) a radiolabel-precursor molecule described herein, or a salt or solvate thereof;
(iii) a composition described herein; or
(iv) a formulation described herein.

Optionally, the kit comprises instructions for use, or one or more reagents for use with the radiolabelled molecule or precursor molecule.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: Schematic depicting exemplary use of radiolabelled molecules of the invention for detecting immune cell enrichment at site of tumour expressing dysfunctional P2X₇ receptor.

FIG. 2: a) Flow cytometry using anti-His antibody to detect CAR-expressing immune cells bound by monomeric E200-Fc fusion protein. X axis: His. b) Flow cytometry using anti-biotin antibody staining from mouse blood and bone marrow via ex vivo incubation of biotinylated monomeric molecule was detectable via anti-biotin antibody to identify the CAR expressing cell subset. X axis: monomeric fusion protein (DetR1, SEQ ID NO: 158).

FIG. 3: Proportion of CD25+/CD69+ and PD-L1+ cells 72 hours following contact with monomeric or dimeric fusion proteins (having the amino acid sequence of SEQ ID NOs: 158 and 149, respectively).

TABLE 1
Sequence information
exemplary sequences of dysfunctional P2X₇ receptor and receptor
epitope moieties

| Peptide/protein | | SEQ ID |
|---|---|---|
| name | Sequence | NO: |
| Full length P2X₇ | MPACCSCSDVFQYETNKVTRIQSMNYGTIKWFFHVIIFSYVCFAL | 1 |
| receptor | VSDKLYQRKEPVISSVHTKVKGIAEVKEEIVENGVKKLVHSVFDT | |
| E200 and E300 | ADYTFPLQGNSFFVMTNFLKTEGQEQRLCPEYPTRRTLCSSDR | |
| shown in bold | GCKKGWMDPQSKGIQTGRCVVYEGNQKTCEVSAWCPIEAVEE | |
| and underline | APRPALLNSAENFTVLIKNNIDFPGHNYTTRNILPGLNITCTFHKT | |
| Pro 210 shown in | QNPQCPIFRLGDIFRETGDNFSDVAIQGGIMGIEIYWDCNLDRWF | |
| italics | HHCRPKYSFRRLDDKTTNVSLYPGYNFRYAKYYKENNVEKRTLI | |
| | KVFGIRFDILVFGTGGKFDIIQLVVYIGSTLSYFGLAAVFIDFLIDTY | |
| | SSNCCRSHIYPWCKCCQPCVVNEYYYRKKCESIVEPKPTLKYVS | |
| | FVDESHIRMVNQQLLGRSLQDVKGQEVPRPAMDFTDLSRLPLAL | |
| | HDTPPIPGQPEEIQLLRKEATPRSRDSPVWCQCGSCLPSQLPES | |
| | HRCLEELCCRKKPGACITTSELFRKLVLSRHVLQFLLLYQEPLLAL | |
| | DVDSTNSRLRHCAYRCYATWRFGSQDMADFAILPSCCRWRIRK | |
| | EFPKSEGQYSGFKSPY | |
| Exemplary E200 | GHNYTTRNILPGLNITC | 2 |
| epitope | | |
| Variant E200 | GHNYTTRNILPGLNIT | 3 |
| epitope (E200′) | | |
| E300 | KYYKENNVEKRTLIK | 4 |
| Variant E300 | KYYKENNVEKRTLIKVF | 5 |
| epitope (E300′) | | |
| Composite | GHNYTTRNILPGAGAKYYKENNVEK | 6 |
| E200/E300 | | |
| epitope | | |
| E200 epitope | GHNYTTRNILPGLNITS | 7 |
| Cys to Ser | | |
| modification | | |
| (“Core” E200 | | |
| sequence) | | |
| Extended E200 | GHNYTTRNILPGLNITSTFHK | 8 |
| Cys to Ser | | |
| modification | | |
| Extended E200′ | GHNYTTRNILPGLNITSTFHKT | 9 |
| Cys to Ser | | |
| modification | | |
| Extended E200″ | GHNYTTRNILPGLNITSTFHKTC | 10 |
| Cys to Ser | | |
| modification | | |
| Extended Pep17 | GHNYTTRNILPGLNITSTFHKTSGSGK | 11 |
| Pep17 | GHNYTTRNILPGLNITSTFHKTS | 12 |
| Extended E200′ | DFPGHNYTTRNILPGLNITSTFHKT | 122 |
| Cys to Ser | | |
| modification + N | | |
| term ext | | |
| Pep16 | DFPGHNYTTRNILPGC | 13 |
| Minimum E200 | NYTTRNILPGL | 14 |
| sequence | | |
| E200_G4S | GHNYTTRNILPGLNITSGGGGS | 15 |
| E200_2xG4S | GHNYTTRNILPGLNITSGGGGSGGGGS | 16 |
| E200_3xG4S | GHNYTTRNILPGLNITSGGGGSGGGGSGGGGS | 17 |
| E200_extended | GHNYTTRNILPGLNITSTFHKTGS | 18 |
| peptide 17v3 (24 | | |
| aa) | | |
| E200_extended | GHNYTTRNILPGLNITSTFHGS | 19 |
| peptide 17v4 (22 | | |
| aa) | | |
| E200_extended | GHNYTTRNILPGLNITSGS | 20 |
| peptide 17v5 (19 | | |
| aa) | | |
| E200_extended | DFPGHNYTTRNILPGLNITSGS | 21 |
| peptide 17v6 (22 | | |
| aa) | | |
| E200_extended | DFPGHNYTTRNILPGLNITSGGGGS | 22 |
| peptide 17v7 (25 | | |
| aa) | | |
| E200_extended | DFPGHNYTTRNILPGLNITSGGGGSGGGGS | 23 |
| peptide 17v8 (30 | | |
| aa) | | |
| E200_extended | DFPGHNYTTRNILPGLNITSGGGGSGGGGSGGGGS | 24 |
| peptide 17v9 (35 | | |
| aa) | | |
| E200_extended | DFPGHNYTTRNILPGLNITSTFHKTSGSGK | 25 |
| peptide 17v10 | | |
| (30 aa) | | |
| E200_extended | DFPGHNYTTRNILPGLNITSTFHKTSGSGKGS | 26 |
| peptide 17v11 | | |
| (32 aa) | | |
| E200_extended | DFPGHNYTTRNILPGLNITSTFHKTSGSGKGGGGS | 27 |
| peptide 17v12 | | |
| (35 aa) | | |
| E200_extended | DFPGHNYTTRNILPGLNITSTFHGGGGS | 28 |
| peptide 17v13 | | |
| (25 aa) | | |
| E200_extended | GHNYTTRNILPGLNITSTFHGGGGS | 29 |
| peptide 17v14 | | |
| (22 aa) | | |
| E200_extended | DFPGHNYTTRNILPGLNITSTFHKTGGGGS | 30 |
| peptide 17v15 | | |
| (30 aa) | | |
| E200_extended | GHNYTTRNILPGLNITSTFHKTGGGGS | 31 |
| peptide 17v16 | | |
| (27 aa) | | |
| E200 + IgG hinge | GHNYTTRNILPGLNITSEPKSSDKTHT | 32 |
| (E200 | | |
| underlined) | | |
| E200 + GS | GHNYTTRNILPGLNITSGSEPKSSDKTHT | 33 |
| linker_IgG hinge | | |
| E200 + G4S | GHNYTTRNILPGLNITSGGGGSEPKSSDKTHT | 34 |
| linker + IgG hinge | | |
| Extended E200 + | GHNYTTRNILPGLNITSTFHKTSGSGKEPKSSDKTHT | 35 |
| _IgG hinge | | |
| Extended E200 + | GHNYTTRNILPGLNITSTFHKTSGSGKGSEPKSSDKTHT | 36 |
| GS linker + _IgG | | |
| hinge | | |
| Extended E200 + | GHNYTTRNILPGLNITSTFHKTSGSGKGGGGSEPKSSDKTHT | 37 |
| G4S linker + IgG | | |
| hinge | | |
| E200 + IgG | GHNYTTRNILPGLNITSEPKSSDKTHTGS | 38 |
| hinge + GS linker | | |
| E200 + GS | GHNYTTRNILPGLNITSGSEPKSSDKTHTGS | 39 |
| linker + IgG | | |
| hinge + GS linker | | |
| E200 + G4Slinker + | GHNYTTRNILPGLNITSGGGGSEPKSSDKTHTGS | 40 |
| IgG hinge + GS | | |
| linker | | |
| Extended | GHNYTTRNILPGLNITSTFHKTSGSGKEPKSSDKTHTGS | 41 |
| E200 + _IgG | | |
| hinge + GSlinker | | |
| Extended | GHNYTTRNILPGLNITSTFHKTSGSGKGSEPKSSDKTHTGS | 42 |
| E200 + GS | | |
| linker + _IgG | | |
| hinge + GS linker | | |
| Extended | GHNYTTRNILPGLNITSTFHKTSGSGKGGGGSEPKSSDKTHTGS | 43 |
| E200 + G4S | | |
| linker + _IgG | | |
| hinge + GS linker | | |
| E200 + IgG | GHNYTTRNILPGLNITSEPKSSDKTHTGGGGS | 44 |
| hinge + G4S linker | | |
| E200 + GS | GHNYTTRNILPGLNITSGSEPKSSDKTHTGGGGS | 45 |
| linker + IgG | | |
| hinge + G4S linker | | |
| E200 + G4S | GHNYTTRNILPGLNITSGGGGSEPKSSDKTHTGGGGS | 46 |
| linker + IgG | | |
| hinge + G4S linker | | |
| Extended | GHNYTTRNILPGLNITSTFHKTSGSGKEPKSSDKTHTGGGGS | 47 |
| E200 + IgG | | |
| hinge + G4S linker | | |
| Extended | GHNYTTRNILPGLNITSTFHKTSGSGKGSEPKSSDKTHTGGGGS | 48 |
| E200 + GS | | |
| linker + IgG | | |
| hinge + G4S linker | | |
| Extended | GHNYTTRNILPGLNITSTFHKTSGSGKGGGGSEPKSSDKTHTGG | 49 |
| E200 + G4S | GGS | |
| linker + IgG | | |
| hinge + G4S linker | | |
| N-term extended | DFPGHNYTTRNILPGLNITSEPKSSDKTHT | 50 |
| E200 + IgG hinge | | |
| N-term extended | DFPGHNYTTRNILPGLNITSGSEPKSSDKTHT | 51 |
| E200 + GS | | |
| linker + IgG hinge | | |
| N-term extended | DFPGHNYTTRNILPGLNITSGGGGSEPKSSDKTHT | 52 |
| E200 + G4S | | |
| linker + IgG hinge | | |
| N and C term | DFPGHNYTTRNILPGLNITSTFHKTSGSGKEPKSSDKTHT | 53 |
| extended | | |
| E200 + IgG hinge | | |
| N and C term | DFPGHNYTTRNILPGLNITSTFHKTSGSGKGSEPKSSDKTHT | 54 |
| extended | | |
| E200 + GS | | |
| linker + IgG hinge | | |
| N and C term | DFPGHNYTTRNILPGLNITSTFHKTSGSGKGGGGSEPKSSDKTHT | 55 |
| extended E200 + | | |
| G4S linker + IgG | | |
| hinge | | |
| N-term extended | DFPGHNYTTRNILPGLNITSEPKSSDKTHTGS | 56 |
| E200 + IgG hinge + | | |
| GS linker | | |
| N-term extended | DFPGHNYTTRNILPGLNITSGSEPKSSDKTHTGS | 57 |
| E200 + GS | | |
| linker + IgG hinge + | | |
| GS linker | | |
| N-term extended | DFPGHNYTTRNILPGLNITSGGGGSEPKSSDKTHTGS | 58 |
| E200 + G4S | | |
| linker + IgG hinge + | | |
| GS linker | | |
| N-term and C | DFPGHNYTTRNILPGLNITSTFHKTSGSGKEPKSSDKTHTGS | 59 |
| term extended | | |
| E200 + lgG | | |
| hinge + GS linker | | |
| N-term and C | DFPGHNYTTRNILPGLNITSTFHKTSGSGKGSEPKSSDKTHTGS | 60 |
| term extended | | |
| E200 + GS | | |
| linker + IgG | | |
| hinge + GS linker | | |
| N-term and C | DFPGHNYTTRNILPGLNITSTFHKTSGSGKGGGGSEPKSSDKTH | 61 |
| term extended | TGS | |
| E200 + G4S | | |
| linker + IgG | | |
| hinge + GS linker | | |
| N-term extended | DFPGHNYTTRNILPGLNITSEPKSSDKTHTGGGGS | 62 |
| E200 + IgG hinge + | | |
| G4S linker | | |
| N-term extended | DFPGHNYTTRNILPGLNITSGSEPKSSDKTHTGGGGS | 63 |
| E200 + GS | | |
| linker + IgG hinge + | | |
| G4S linker | | |
| N-term extended | DFPGHNYTTRNILPGLNITSGGGGSEPKSSDKTHTGGGGS | 64 |
| E200 + G4S | | |
| linker + IgG hinge + | | |
| G4S linker | | |
| N-term and C | DFPGHNYTTRNILPGLNITSTFHKTSGSGKEPKSSDKTHTGGGGS | 65 |
| term extended | | |
| E200 + IgG | | |
| hinge + G4S linker | | |
| N-term and C | DFPGHNYTTRNILPGLNITSTFHKTSGSGKGSEPKSSDKTHTGG | 66 |
| term extended | GGS | |
| E200 + GS | | |
| linker + IgG | | |
| hinge + G4S linker | | |
| N-term and C | DFPGHNYTTRNILPGLNITSTFHKTSGSGKGGGGSEPKSSDKTH | 67 |
| term extended | TGGGGS | |
| E200 + G4S | | |
| linker + IgG | | |
| hinge + G4S linker | | |
| IgG hinge + G4S | DKTHTSPPSPAPELLGGGGSDFPGHNYTTRNILPGLNITS | 68 |
| linker + N-term | | |
| extended E200 | | |
| (underlined) | | |
| G4S linker + IgG | GGGGSEPKSSDKTHTSPPSPAPELLGGGGSDFPGHNYTTRNILP | 69 |
| hinge+N-term | GLNITS | |
| extended E200 | | |
| (underlined) | | |
| G4S linker | GGGGS | 70 |
| IgG hinge region | EPKSSDKTHT | 71 |
| linker | | |
| GS linker + IgG | GSEPKSSDKTHTSPPSPAPELL | 72 |
| hinge | | |
| G4S linker + IgG | GGGGSEPKSSDKTHTSPPSPAPELLGGGGS | 73 |
| hinge + G4S | | |
| IgG hinge + GS | EPKSSDKTHTGS | 74 |
| linker | | |
| IgG hinge + G4S | EPKSSDKTHTGGGGS | 75 |
| linker | | |
| Exemplary C | TFHKT | 138 |
| terminal | | |
| extension of | | |
| E200 epitope | | |
| Exemplary N | DFP | 139 |
| terminal | | |
| extension of | | |
| E200 epitope | | |
| N terminal | DFPGHNYTTRNILPGLNITS | 140 |
| extended core E | | |
| 200 epitope | | |
| Exemplary IgG1 | EPKSCDKTHTSPPSPAP | 141 |
| hinge region for | | |
| monomeric | | |
| fusion proteins | | |
| (two C to S | | |
| mutations) | | |
| Exemplary IgG1 | EPKSSDKTHTSPPSPAP | 142 |
| hinge region for | | |
| monomeric | | |
| fusion proteins | | |
| (three C to S | | |
| mutations) | | |
| Exemplary G4S | GGGGS | 143 |
| linker sequence | | |
| Exemplary | LEVLFQGPVRR | 144 |
| cleavable linker | | |
| (protease | | |
| recognition site; | | |
| cleavage | | |
| between Q and | | |
| G residues) | | |
| Exemplary E200- | DFPGHNYTTRNILPGLNITSTFHKTSGSGKGGGGSEPKSSDKTH | 145 |
| Fc fusion | TSPPSPAPPVAGPSVFLFPPKPKDTLMISRTPEVTCVVVGVSHED | |
| sequence | PEVKFNWYVDGVEVHNAKTKPREEQYQSTYRVVSVLTVLHQDW | |
| “DetR1” | LNGKEYKCKVSNKQLPSPIEKTISKAKGQPREPQVYTLPPSRDEL | |
| (monomeric and | TKNQVSLTCLVKGFYPSDIAVEWESNGQPENNYKTTPPVLDSDG | |
| Fc attenuated: | SFFLYSKLTVDKSRWQQGNVFSCSVMHEALHNHYTQKSLSLSP | |
| E233P; L234V; | GK | |
| L235A; ΔG236, | | |
| D265G, N297Q, | | |
| A327Q, A330S | | |
| shown in italics | | |
| and underline) | | |
| (E200 moiety | | |
| underlined; linker | | |
| in bold) | | |
| Exemplary E200- | GHNYTTRNILPGLNITSTFHKTSGSGKGGGGSEPKSCDKTHTSP | 146 |
| Fc fusion | PSPAPPVAGPSVFLFPPKPKDTLMISRTPEVTCVVVGVSHEDPE | |
| sequence | VKFNWYVDGVEVHNAKTKPREEQYQSTYRVVSVLTVLHQDWLN | |
| “DetR2” | GKEYKCKVSNKQLPSPIEKTISKAKGQPREPQVYTLPPSRDELTK | |
| (monomeric and | NQVSLTCLVKGFYPSDIAVEWESNGQPENNYKTTPPVLDSDGSF | |
| Fc attenuated) | FLYSKLTVDKSRWQQGNVFSCSVMHEALHNHYTQKSLSLSPGK | |
| (E200 moiety | | |
| underlined; linker | | |
| in bold; cys to | | |
| serine in italics | | |
| Exemplary E200- | DFPGHNYTTRNILPGLNITSTFHKTSGSGKGGGGSEPKSSDKTH | 147 |
| Fc fusion | TSPPAPAPEAARGPSVFLFPPKPKDTLMISRTPEVTCVVVDVSHE | |
| sequence | DPEVKFNWYVDGVEVHNAKTKPREEQYNSTYRVVSVLTVLHQD | |
| “DetR1” + | WLNGKEYKCKVSNKALPAPIEKTISKAKGQPREPQVYTLPPSRD | |
| LALA + G236R | ELTKNQVSLTCLVKGFYPSDIAVEWESNGQPENNYKTTPPVLDS | |
| (monomeric and | DGSFFLYSKLTVDKSRWQQGNVFSCSVMHEALHNHYTQKSLSL | |
| Fc attenuated) | SPGK | |
| (E200 moiety | | |
| underlined; linker | | |
| in bold; cys to | | |
| serine in italics; | | |
| LALA + c1q | | |
| mutations in | | |
| italics and | | |
| underline) | | |
| Exemplary E200- | GHNYTTRNILPGLNITSTFHKTSGSGKGGGGSEPKSSDKTHTSP | 148 |
| Fc fusion | PSPAPEAARGPSVFLFPPKPKDTLMISRTPEVTCVVVDVSHEDP | |
| sequence | EVKFNWYVDGVEVHNAKTKPREEQYNSTYRVVSVLTVLHQDWL | |
| “DetR2” + | NGKEYKCKVSNKALPAPIEKTISKAKGQPREPQVYTLPPSRDELT | |
| LALA + G236R | KNQVSLTCLVKGFYPSDIAVEWESNGQPENNYKTTPPVLDSDGS | |
| (monomeric and | FFLYSKLTVDKSRWQQGNVFSCSVMHEALHNHYTQKSLSLSPGK | |
| Fc attenuated) | | |
| (E200 moiety | | |
| underlined; linker | | |
| in bold; cys to | | |
| serine in italics; | | |
| LALA + C1q | | |
| mutations in | | |
| italics and | | |
| underline) | | |
| Exemplary E200- | DFPGHNYTTRNILPGLNITSTFHKTSGSGKGGGGSEPKSCDKTH | 149 |
| Fc fusion | TCPPCPAPPVAGPSVFLFPPKPKDTLMISRTPEVTCVVVGVSHE | |
| sequence | DPEVKFNWYVDGVEVHNAKTKPREEQYQSTYRVVSVLTVLHQD | |
| “DetR1” | WLNGKEYKCKVSNKQLPSPIEKTISKAKGQPREPQVYTLPPSRD | |
| (Fc attenuated) | ELTKNQVSLTCLVKGFYPSDIAVEWESNGQPENNYKTTPPVLDS | |
| (E200 moiety | DGSFFLYSKLTVDKSRWQQGNVFSCSVMHEALHNHYTQKSLSL | |
| underlined; linker | SPGK | |
| in bold) | | |
| Exemplary E200- | GHNYTTRNILPGLNITSTFHKTSGSGKGGGGSEPKSCDKTHTCP | 150 |
| Fc fusion | PCPAPPVAGPSVFLFPPKPKDTLMISRTPEVTCVVVGVSHEDPE | |
| sequence | VKFNWYVDGVEVHNAKTKPREEQYQSTYRVVSVLTVLHQDWLN | |
| “DetR2” | GKEYKCKVSNKQLPSPIEKTISKAKGQPREPQVYTLPPSRDELTK | |
| (dimeric Fc | NQVSLTCLVKGFYPSDIAVEWESNGQPENNYKTTPPVLDSDGSF | |
| attenuated) | FLYSKLTVDKSRWQQGNVFSCSVMHEALHNHYTQKSLSLSPGK | |
| (E200 moiety | | |
| underlined; linker | | |
| in bold) | | |
| Exemplary E200- | DFPGHNYTTRNILPGLNITSTFHKTSGSGKGGGGSEPKSSDKTH | 151 |
| Fc fusion | TCPPCPAPEAARGPSVFLFPPKPKDTLMISRTPEVTCVVVDVSH | |
| sequence | EDPEVKFNWYVDGVEVHNAKTKPREEQYNSTYRVVSVLTVLHQ | |
| “DetR1” + | DWLNGKEYKCKVSNKALPAPIEKTISKAKGQPREPQVYTLPPSR | |
| LALA + G236R | DELTKNQVSLTCLVKGFYPSDIAVEWESNGQPENNYKTTPPVLD | |
| (dimeric Fc | SDGSFFLYSKLTVDKSRWQQGNVFSCSVMHEALHNHYTQKSLS | |
| attenuated) | LSPGK | |
| (E200 moiety | | |
| underlined; linker | | |
| in bold; | | |
| LALA + C1q | | |
| mutations in | | |
| italics and | | |
| underline) | | |
| Exemplary E200- | GHNYTTRNILPGLNITSTFHKTSGSGKGGGGSEPKSSDKTHTCP | 152 |
| Fc fusion | PCPAPEAARGPSVFLFPPKPKDTLMISRTPEVTCVVVDVSHEDP | |
| sequence | EVKFNWYVDGVEVHNAKTKPREEQYNSTYRVVSVLTVLHQDWL | |
| “DetR2” + | NGKEYKCKVSNKALPAPIEKTISKAKGQPREPQVYTLPPSRDELT | |
| LALA + G236R | KNQVSLTCLVKGFYPSDIAVEWESNGQPENNYKTTPPVLDSDGS | |
| (dimeric Fc | FFLYSKLTVDKSRWQQGNVFSCSVMHEALHNHYTQKSLSLSPGK | |
| attenuated) | | |
| (E200 moiety | | |
| underlined; linker | | |
| in bold; | | |
| LALA + C1q | | |
| mutations in | | |
| italics and | | |
| underline) | | |
| Exemplary E200- | DFPGHNYTTRNILPGLNITSTFHKTSGSGKGGGGSLEVLFQGPV | 153 |
| Fc fusion | RREPKSSDKTHTSPP SPAPPVAGPSVFLFPPKPKDTLMISRTPEV | |
| sequence | TCVVVGVSHEDPEVKFNWYVDGVEVHNAKTKPREEQYQSTYRV | |
| “DetR1” | VSVLTVLHQDWLNGKEYKCKVSNKQLPSPIEKTISKAKGQPREP | |
| With cleavable | QVYTLPPSRDELTKNQVSLTCLVKGFYPSDIAVEWESNGQPENN | |
| sequence | YKTTPPVLDSDGSFFLYSKLTVDKSRWQQGNVFSCSVMHEALH | |
| betweem Fc | NHYTQKSLSLSPGK | |
| region and E200 | | |
| moiety | | |
| (monmeric Fc | | |
| attenuated) | | |
| (E200 moiety | | |
| underlined; linker | | |
| in bold; cleavable | | |
| linker in italics; | | |
| cys to serine | | |
| subs in italics) | | |
| Exemplary E200- | DFPGHNYTTRNILPGLNITSTFHKTSGSGKGGGGSLEVLFQGPV | 154 |
| Fc fusion | RREPKSSDKTHTCPPCPAPPVAGPSVFLFPPKPKDTLMISRTPE | |
| sequence | VTCVVVGVSHEDPEVKFNWYVDGVEVHNAKTKPREEQYQSTYR | |
| “DetR1” | VVSVLTVLHQDWLNGKEYKCKVSNKQLPSPIEKTISKAKGQPRE | |
| With cleavable | PQVYTLPPSRDELTKNQVSLTCLVKGFYPSDIAVEWESNGQPEN | |
| sequence | NYKTTPPVLDSDGSFFLYSKLTVDKSRWQQGNVFSCSVMHEAL | |
| between Fc | HNHYTQKSLSLSPGK | |
| region and E200 | | |
| moiety | | |
| dimeric Fc | | |
| attenuated) | | |
| (E200 moiety | | |
| underlined; linker | | |
| in bold; cleavable | | |
| linker in italics) | | |
| Exemplary E200- | DFPGHNYTTRNILPGLNITSTFHKTSGSGKGGGGSEPKSSDKTH | 155 |
| Fc fusion | TSPPSPAPEAARGPSVFLFPPKPKDTLMASRTPEVTCVVVDVSH | |
| sequence | EDPEVKFNWYVDGVEVHNAKTKPREEQYNSTYRVVSVLTVLAQ | |
| “DetR1” + | DWLNGKEYKCKVSNKALPAPIEKTISKAKGQPREPQVYTLPPSR | |
| LALA + G236R: | DELTKNQVSLTCLVKGFYPSDIAVEWESNGQPENNYKTTPPVLD | |
| I253A/H310A | SDGSFFLYSKLTVDKSRWQQGNVFSCSVMHEALHNHYTQKSLS | |
| (monomeric, Fc | LSPGK | |
| attenuated and | | |
| FcRN | | |
| attenuated) | | |
| (E200 moiety | | |
| underlined; linker | | |
| in bold; | | |
| LALA + C1q | | |
| mutations in | | |
| italics and | | |
| underline; | | |
| cysteine | | |
| substitutions in | | |
| italics; | | |
| substitutuions for | | |
| reducing FcRN | | |
| binding in bold | | |
| and underline) | | |
| Exemplary E200- | DFPGHNYTTRNILPGLNITSTFHKTSGSGKGGGGSEPKSSDKTH | 156 |
| Fc fusion | TCPPCPAPEAARGPSVFLFPPKPKDTLMASRTPEVTCVVVDVSH | |
| sequence | EDPEVKFNWYVDGVEVHNAKTKPREEQYNSTYRVVSVLTVLAQ | |
| “DetR1” + | DWLNGKEYKCKVSNKALPAPIEKTISKAKGQPREPQVYTLPPSR | |
| LALA + G236R: | DELTKNQVSLTCLVKGFYPSDIAVEWESNGQPENNYKTTPPVLD | |
| I253A/H310A | SDGSFFLYSKLTVDKSRWQQGNVFSCSVMHEALHNHYTQKSLS | |
| dimeric, Fc | LSPGK | |
| attenuated and | | |
| FcRN | | |
| attenuated) | | |
| (E200 moiety | | |
| underlined; linker | | |
| in bold; | | |
| LALA + C1q | | |
| mutations in | | |
| italics and | | |
| underline;; | | |
| substitutions for | | |
| reducing FcRN | | |
| binding in bold | | |
| and underline) | | |
| Exemplary E200- | DFPGHNYTTRNILPGLNITSTFHKTSGSGKGGGGSEPKSSDKTH | 157 |
| Fc fusion | TCPPCPAPPVAGPSVFLFPPKPKDTLMISRTPEVTCVVVGVSHE | |
| sequence | DPEVKFNWYVDGVEVHNAKTKPREEQYQSTYRVVSVLTVLHQD | |
| “DetR1” | WLNGKEYKCKVSNKQLPSPIEKTISKAKGQPREPQVYTLPPSRD | |
| (Fc attenuated) | ELTKNQVSLTCLVKGFYPSDIAVEWESNGQPENNYKTTPPVLDS | |
| (E200 moiety | DGSFFLYSKLTVDKSRWQQGNVFSCSVMHEALHNHYTQKSLSL | |
| underlined; linker | SPGK | |
| in bold) | | |
| “Knob” Fc region | | |
| Exemplary E200- | DFPGHNYTTRNILPGLNITSTFHKTSGSGKGGGGSEPKSSDKTH | 158 |
| Fc fusion | TSPPSPAPEAAGGPSVFLFPPKPKDTLMISRTPEVTCVVVDVSHE | |
| sequence | DPEVKFNWYVDGVEVHNAKTKPREEQYNSTYRVVSVLTVLHQD | |
| “DetR1” | WLNGKEYKCKVSNKALGAPIEKTISKAKGQPREPQVYTLPPSRK | |
| (Fc attenuated) | ELTKNQVSLTCLVKGFYPSDIAVEWESNGQPENNYKTTPPVLKS | |
| (E200 moiety | DGSFFLYSKLTVDKSRWQQGNVFSCSVMHEALHNHYTQKSLSL | |
| underlined; linker | SPGK | |
| in bold) | | |
| “Knob” Fc region | | |
| (no dimerisation | | |
| due to C to S | | |
| substitutions in | | |
| hinge region | | |
| “Hole” Fc region | EPKSSDKTHTCPPCPAPEAAGGPSVFLFPPKPKDTLMISRTPEVT | 159 |
| (no E200 moiety) | CVVVDVSHEDPEVKFNWYVDGVEVHNAKTKPREEQYNSTYRVV | |
| | SVLTVLHQDWLNGKEYKCKVSNKALGAPIEKTISKAKGQPREPQ | |
| | VYTLPPSRDELTKNQVSLTCLVKGFYPSDIAVEWESNGQPENNY | |
| | DTTPPVLDSDGSFFLYSDLTVDKSRWQQGNVFSCSVMHEALHN | |
| | HYTQKSLSLSPGK | |
| Exemplary E200- | DFPGHNYTTRNILPGLNITSTFHKTSGSGKGGGGSEPKSSDKTH | 160 |
| Fc fusion | TCPPCPAPEAAGGPSVFLFPPKPKDTLMISRTPEVTCVVVDVSH | |
| sequence | EDPEVKFNWYVDGVEVHNAKTKPREEQYNSTYRVVSVLTVLHQ | |
| “DetR1” | DWLNGKEYKCKVSNKALGAPIEKTISKAKGQPREPQVYTLPPSR | |
| (Fc attenuated) | DELTKNQVSLTCLVKGFYPSDIAVEWESNGQPENNYDTTPPVLD | |
| (E200 moiety | SDGSFFLYSDLTVDKSRWQQGNVFSCSVMHEALHNHYTQKSLS | |
| underlined; linker | LSPGK | |
| in bold) | | |
| “Hole” Fc region | | |
| Exemplary E200- | DFPGHNYTTRNILPGLNITSTFHKTSGSGKGGGGSEPKSSDKTH | 161 |
| Fc fusion | TCPPCPAPEAAGGPSVFLFPPKPKDTLMISRTPEVTCVVVDVSH | |
| sequence | EDPEVKFNWYVDGVEVHNAKTKPREEQYNSTYRVVSVLTVLHQ | |
| “DetR1” | DWLNGKEYKCKVSNKALGAPIEKTISKAKGQPREPQVYTLPPSR | |
| (Fc attenuated) | DELTKNQVSLTCLVKGFYPSDIAVEWESNGQPENNYDTTPPVLD | |
| (E200 moiety | SDGSFFLYSDLTVDKSRWQQGNVFSCSVMHEALHNHYTQKSLS | |
| underlined; linker | LSPGK | |
| in bold) | | |
| “Hole” Fc region | | |
| (no dimerisation | | |
| due to c to S | | |
| substitutions in | | |
| hinge region | | |
| “Knob” Fc region | EPKSSDKTHTSPPSPAPEAAGGPSVFLFPPKPKDTLMISRTPEVT | 162 |
| (no E200 moiety) | CVVVDVSHEDPEVKFNWYVDGVEVHNAKTKPREEQYNSTYRVV | |
| | SVLTVLHQDWLNGKEYKCKVSNKALGAPIEKTISKAKGQPREPQ | |
| | VYTLPPSRKELTKNQVSLTCLVKGFYPSDIAVEWESNGQPENNY | |
| | KTTPPVLKSDGSFFLYSKLTVDKSRWQQGNVFSCSVMHEALHN | |
| | HYTQKSLSLSPGK | |

### DETAILED DESCRIPTION OF THE EMBODIMENTS

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognise many methods and materials similar or equivalent to those described herein, which could be used in the practice of the present invention. The present invention is in no way limited to the methods and materials described.

It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

The present invention provides a radiolabelled molecule, compositions and kits comprising the same, that may be useful for detecting an immune cell that expresses a receptor and signalling domain, such as a CAR T cell. Further, the invention provides a radiolabel-precursor molecule that can be used to generate said radiolabelled molecule.

The radiolabelled molecule comprises (i) a dysfunctional P2X₇ receptor epitope moiety that is recognised or capable of being bound by an antigen recognition domain of a receptor expressed on an immune cell, wherein the receptor is for binding a dysfunctional P2X₇ receptor and comprises a signalling domain and (ii) a radionuclide that is linked to the epitope moiety. The epitope moiety allows for specific binding of the radiolabelled molecule to the receptor expressed by the immune cell, and the radionuclide allows for detection of the radiolabelled molecule, and consequently the location and/or distribution of immune cells expressing the receptor.

The radiolabelled molecule may therefore advantageously provide a new approach for real time monitoring of the distribution and/or quantification of immune cells, such as CAR T cells, for binding dysfunctional P2X₇ receptor, in vivo.

In particularly preferred embodiments of the invention, there are provided fusion proteins or heterodimeric molecules derived therefrom, comprising a linear epitope derived from the P2X₇ receptor (such as exemplified in any of SEQ ID NOs: 14 or 7) and an Fc region of an antibody. The presence of an Fc region in the fusion proteins provides for particular advantages including in relation to preventing loss of the reagent from the circulation due to renal filtration. Thus, the molecules of the invention preferably comprise an Fc region from an antibody to assist with stability of the protein in the circulation of the subject for whom detection of CAR T cells is to be determined.

In especially preferred embodiments, the Fc fusion proteins are designed so as to comprise only a single copy of the linear epitope derived from the P2X₇ receptor. This can be accomplished, as further described herein, by introducing amino acid substitutions into the Fc region to prevent homodimerisation, or alternatively, using the well-known knob-into-holes technology for ensuring formation of an asymmetric heterodimeric molecule (eg comprising a E200 peptide-Fc fusion protein and an Fc region that does not comprise an E200 peptide). Such monomeric or asymmetric heterodimeric molecules have the advantage of reducing activation of the target immune cells and preventing unwanted exhaustion of the target immune cells (as further described herein in the examples). Without wishing to be bound by theory, the inventors believe that this is due to the reduced ability of the molecules to cross-link either two different CAR receptors on one cell or two different CAR receptors on two separate CAR expressing cells.

### DEFINITIONS

Unless defined otherwise, technical and scientific terms used herein have the same meaning as commonly understood by one of ordinary skill in the art to which this invention belongs.

For purposes of interpreting this specification, the following definitions will generally apply and whenever appropriate, terms used in the singular will also include the plural and vice versa.

As used herein, the term “and/or”, e.g., “X and/or Y” will be understood to mean either “X and Y” or “X or Y” and shall be taken to provide explicit support for both meanings or for either meaning.

The articles “a” and “an” are used herein to refer to one or to more than one (i.e. to at least one) of the grammatical object of the article. By way of example, “a dysfunctional P2X₇ receptor epitope moiety” means one dysfunctional P2X₇ receptor epitope moiety or more than one dysfunctional P2X₇ receptor epitope moiety.

As used herein, except where the context requires otherwise, the term “comprise” and variations of the term, such as “comprising”, “comprises” and “comprised”, are not intended to exclude further additives, components, integers or steps.

“Purinergic receptor” generally refers to a receptor that uses a purine (such as ATP) as a ligand.

“P2X₇ receptor” generally refers to a purinergic receptor formed from three protein subunits or monomers, with at least one of the monomers having an amino acid sequence substantially as shown in SEQ ID NO: 1 in Table 1 herein.

To the extent that P2X₇ receptor is formed from three monomers, it is a “trimer” or “trimeric”. “P2X₇ receptor” encompasses naturally occurring variants of P2X₇ receptor, e.g., wherein the P2X₇ monomers are splice variants, allelic variants, SNPs and isoforms including naturally-occurring truncated or secreted forms of the monomers forming the P2X₇ receptor (e.g., a form consisting of the extracellular domain sequence or truncated form of it), naturally-occurring variant forms (e.g., alternatively spliced forms) and naturally-occurring allelic variants. In certain embodiments of the invention, the native sequence P2X₇ monomeric polypeptides disclosed herein are mature or full-length native sequence polypeptides comprising the full-length amino acids sequence shown in SEQ ID NO: 1. In certain embodiments the P2X₇ receptor may have an amino acid sequence that is modified, for example various of the amino acids in the sequence shown in SEQ ID NO: 1 may be substituted, deleted, or a residue may be inserted.

“Functional P2X₇ receptor” generally refers to a form of the P2X₇ receptor having three intact binding sites or clefts for binding to ATP. When bound to ATP, the functional receptor forms a non-selective sodium/calcium channel that converts to a pore-like structure that enables the ingress of calcium ions and molecules of up to 900 Da into the cytosol, one consequence of which may be induction of programmed cell death. In normal homeostasis, expression of functional P2X₇ receptors is generally limited to cells that undergo programmed cell death such as thymocytes, dendritic cells, lymphocytes, macrophages and monocytes. There may also be some expression of functional P2X₇ receptors on erythrocytes and other cell types.

“Dysfunctional P2X₇ receptor” (also called “non-functional” or (nf) P2X₇) is a P2X₇ receptor that has an impaired response to ATP such that it is unable to form an apoptotic pore under physiological conditions. A dysfunctional P2X₇ receptor or (nfP2X₇ receptor) generally refers to a form of a P2X₇ receptor having a conformation, distinct from functional P2X₇, whereby the receptor is unable to form an apoptotic pore, but which is still able to operate as a non-selective channel through the maintenance of a single functional ATP binding site located between adjacent monomers. One example arises where one or more of the monomers has a cis isomerisation at Pro210 (according to SEQ ID NO: 1). The isomerisation may arise from any molecular event that leads to misfolding of the monomer, including for example, mutation of monomer primary sequence or abnormal post translational processing. One consequence of the isomerisation is that the receptor is unable to bind to ATP at one, or more particularly two, ATP binding sites on the trimer and as a consequence not be able to extend the opening of the channel. In the circumstances, the receptor cannot form a pore and this limits the extent to which calcium ions may enter the cytosol. Dysfunctional P2X₇ receptors are expressed on a wide range of epithelial and haematopoietic cancers. As used herein, the term “dysfunctional P2X₇ receptors” may be used interchangeably with the term “non-functional P2X₇ receptors” or “nfP2X₇ receptors”.

“Cancer associated-P2X₇ receptors” are generally P2X₇ receptors that are found on cancer cells (including, pre-neoplastic, neoplastic, malignant, benign or metastatic cells), but not on non-cancer or normal cells.

“E200 epitope” generally refers to an epitope having the sequence GHNYTTRNILPGLNITC (SEQ ID NO: 2). Variants thereof are exemplified in Table 1 and include any of SEQ ID NOs: 3, or 7 to 69 or 155.

“E300 epitope” generally refers to an epitope having the sequence KYYKENNVEKRTLIK (SEQ ID NO: 4) or a variant thereof, as defined in SEQ ID NO: 5.

A “composite epitope” generally refers to an epitope that is formed from the juxtaposition of the E200 and E300 epitopes or parts of these epitopes. An example of a composite epitope comprising E200 and E300 epitopes is GHNYTTRNILPGAGAKYYKENNVEK (SEQ ID NO: 6).

As used herein, the term “antigen” is intended to include substances that bind to or evoke the production of one or more antibodies and may comprise, but is not limited to, proteins, peptides, polypeptides, oligopeptides, lipids, carbohydrates, and combinations thereof, for example a glycosylated protein or a glycolipid. The term “antigen” as used herein refers to a molecular entity that may be expressed on a target cell and that can be recognised by means of the adaptive immune system including but not restricted to antibodies or TCRs, or engineered molecules including but not restricted to transgenic TCRs, CARs, scFvs or multimers thereof, Fab-fragments or multimers thereof, antibodies or multimers thereof, single chain antibodies or multimers thereof, or any other molecule that can execute binding to a structure with high affinity.

“Epitope” generally refers to that part of an antigen that is bound by the antigen binding site of an antibody. An epitope may be “linear” in the sense that the hypervariable loops of the antibody CDRs that form the antigen binding site bind to a sequence of amino acids as in a primary protein structure. In certain embodiments, the epitope is a “conformational epitope” i.e. one in which the hypervariable loops of the CDRs bind to residues as they are presented in the tertiary or quaternary protein structure.

The terms “binds to”, “specifically binds to” or “specific for” with respect to a receptor referring to an antigen-binding domain that recognises and binds a dysfunctional P2X₇ receptor, is intended to mean that the receptor does not substantially recognise or bind to other antigens in a sample.

The terms “immune cell” or “immune effector cell” refer to a cell that may be part of the immune system and executes a particular effector function such as alpha-beta T cells, NK cells, NKT cells, B cells, Breg cells, Treg cells, innate lymphoid cells (ILC), cytokine induced killer (CIK) cells, lymphokine activated killer (LAK) cells, gamma-delta T cells, mesenchymal stem cells or mesenchymal stromal cells (MSC), monocytes or macrophages or any hematopoietic progenitor cells such as pluripotent stem cells and early progenitor subsets that may mature or differentiate into somatic cells. The cells may be naturally occurring or generated by cytokine exposure, artificial/genetically modified cells (such as iPSCs and other artificial cell types). Preferred immune cells are cells with cytotoxic effector function such as alpha-beta T cells, NK cells, NKT cells, ILC, ClK cells, LAK cells or gamma-delta T cells. “Effector function” means a specialised function of a cell, e.g. in a T cell an effector function may be cytolytic activity or helper cell activity including the secretion of cytokines.

As used herein, the term “amino acid” refers to a compound having an amino group and a carboxylic acid group. The amino acid may be a L- or D-isomer or mixtures thereof. The amino acid may have a naturally occurring side chain (see Table 1) or a non-proteinogenic side chain. The amino acid may also be further substituted in the α-position with a group selected from —C₁₋₆ alkyl, —(CH₂)ₙCORₐ, —(CH₂)ₙR_{b} and —PO₃H, where n is an integer selected from 1 to 8, Rₐ is —OH, —NH₂, —NHC₁₋₃ alkyl, —OC₁₋₃ alkyl or —C₁₋₃ alkyl and R_{b} is —OH, —SH, —SC₁₋₃ alkyl, —OC₁₋₃ alkyl, —NH₂, —NHC₁₋₃ alkyl or —NHC(C═NH)NH₂ and where each alkyl group may be substituted with one or more groups selected from —OH, —NH₂, —NHC₁₋₃ alkyl, —OC₁₋₃ alkyl, —SH, —SC₁₋₃ alkyl, —CO₂H, —CO₂C₁₋₃ alkyl, —CONH₂ and —CONHC₁₋₃ alkyl.

Amino acid structure and single and three letter abbreviations used throughout the specification are defined in Table 2, which lists the twenty proteinogenic naturally occurring amino acids which occur in proteins as L-isomers.

TABLE 2 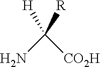
(1) 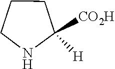
(2)

| | Three-letter | One-letter | Structure of side chain |
|---|---|---|---|
| Amino Acid | Abbreviation | symbol | (R) |
| Alanine | Ala | A | —CH₃ |
| Arginine | Arg | R | —(CH₂)₃NHC(═N)NH₂ |
| Asparagine | Asn | N | —CH₂CONH₂ |
| Aspartic acid | Asp | D | —CH₂CO₂H |
| Cysteine | Cys | C | —CH₂SH |
| Glutamine | Gln | Q | —(CH₂)₂CONH₂ |
| Glutamic acid | Glu | E | —(CH₂)₂CO₂H |
| Glycine | Gly | G | —H |
| Histidine | His | H | —CH₂(4-imidazolyl) |
| Isoleucine | Ile | I | —CH(CH₃)CH₂CH₃ |
| Leucine | Leu | L | —CH₂CH(CH₃)₂ |
| Lysine | Lys | K | —(CH₂)₄NH₂ |
| Methionine | Met | M | —(CH₂)₂SCH₃ |
| Phenylalanine | Phe | F | —CH₂Ph |
| Proline | Pro | P | see formula (2) above for |
| | | | structure of amino acid |
| Serine | Ser | S | —CH₂OH |
| Threonine | Thr | T | —CH(CH₃)OH |
| Tryptophan | Trp | W | —CH₂(3-indolyl) |
| Tyrosine | Tyr | Y | —CH₂(4-hydroxyphenyl) |
| Valine | Val | V | —CH(CH₃)₂ |

As used herein, the term “non-proteinogenic amino acid” refers to an amino acid having a side chain that does not occur in the naturally occurring L-α-amino acids recited in Table 2. Examples of non-proteinogenic amino acids and derivatives include, but are not limited to, norleucine, 4-aminobutyric acid, 4-amino-3-hydroxy-5-phenylpentanoic acid, 6-aminohexanoic acid, t-butylglycine, norvaline, phenylglycine, ornithine, citrulline, sarcosine, 4-amino-3-hydroxy-6-methylheptanoic acid, 2-thienyl alanine and/or D-isomers of natural amino acids

As used herein, the term “α-amino acid” refers to an amino acid that has a single carbon atom (the α-carbon atom) separating a carboxyl terminus (C-terminus) and an amino terminus (N-terminus). An α-amino acid includes naturally occurring and non-naturally occurring L-amino acids and their D-isomers and derivatives thereof such as salts or derivatives where functional groups are protected by suitable protecting groups. Unless otherwise stated, the term “amino acid” as used herein refers to an α-amino acid.

The term “alkyl” refers to a straight chain or branched saturated hydrocarbon group having 1 to 6 carbon atoms. Where appropriate, the alkyl group may have a specified number of carbon atoms, for example, C₁₋₆ alkyl which includes alkyl groups having 1, 2, 3, 4, 5 or 6 carbon atoms in a linear or branched arrangement. Examples of suitable alkyl groups include, but are not limited to, methyl, ethyl, n-propyl, i-propyl, n-butyl, i-butyl, t-butyl, n-pentyl, 2-methylbutyl, 3-methylbutyl, 4-methylbutyl, n-hexyl, 2-methylpentyl, 3-methylpentyl, 4-methylpentyl, and 5-methylpentyl.

Suitable salts include, but are not limited to, salts of pharmaceutically acceptable inorganic acids such as hydrochloric, sulphuric, phosphoric, nitric, carbonic, boric, sulphamic, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulphonic, toluenesulphonic, benzenesulphonic, salicylic, sulphanilic, aspartic, glutamic, edetic, stearic, palmitic, oleic, lauric, pantothenic, tannic, ascorbic and valeric acids.

Base salts include, but are not limited to: those formed with pharmaceutically acceptable cations, such as: sodium, potassium, lithium, calcium, magnesium, zinc, ammonium and alkylammonium; salts formed from triethylamine; alkoxyammonium salts such as those formed with ethanolamine; and salts formed from ethylenediamine, choline or amino acids such as arginine, lysine or histidine.

Basic nitrogen-containing groups may be quarternised with such agents as lower alkyl halide, such as methyl, ethyl, propyl, and butyl chlorides, bromides and iodides; dialkyl sulphates like dimethyl and diethyl sulphate; and others.

Salts, or other derivatives of the compounds of the present invention may be provided in the form of solvates. Solvates contain either stoichiometric or non-stoichiometric amounts of a solvent, and may be formed during the process of crystallization with pharmaceutically acceptable solvents such as water, alcohols such as methanol, ethanol or isopropyl alcohol, DMSO, acetonitrile, dimethyl formamide (DMF) and the like with the solvate forming part of the crystal lattice by either non-covalent binding or by occupying a hole in the crystal lattice. Hydrates are formed when the solvent is water, alcoholates are formed when the solvent is alcohol. Solvates of the compounds of the present invention can be conveniently prepared or formed during the processes described herein. In general, the solvated forms are considered equivalent to the unsolvated forms for the purposes of the compounds and methods provided herein.

As used herein, the term “subject” refers to a mammal such as mouse, rat, cow, pig, goat, chicken, dog, monkey or human. Preferentially, the subject is a human. The subject may be a subject suffering from a disorder such as cancer (a patient), but the subject also may be a healthy subject.

### Radiolabelled Molecules

It will be appreciated that a radiolabelled molecule of the invention may be in any form, provided that it comprises (i) a dysfunctional P2X₇ receptor epitope moiety that is recognised or capable of being bound by an antigen binding domain of a receptor for binding a dysfunctional P2X₇ receptor expressed on an immune cell, wherein the receptor comprises a signalling domain; and (ii) a radionuclide that is directly or indirectly linked to the epitope moiety.

Typically, the dysfunctional P2X₇ receptor epitope moiety is in the form of a peptide. The dysfunctional P2X₇ receptor epitope moiety is further described herein.

It will be appreciated that the presence of a radionuclide may affect the local charge field of nearby atoms. Therefore, it may be preferable to link the radionuclide at a location in the radiolabelled molecule such that the radionuclide does not affect the binding and recognition of the epitope moiety by the antigen binding domain of the receptor.

Accordingly, in some embodiments, the epitope moiety comprises one or more spacers between the dysfunctional P2X₇ receptor epitope moiety and the radionuclide. A spacer is an amino acid sequence in the epitope moiety which may be present N or C terminal to the recognition sequence of the epitope moiety. In the case that a radionuclide that is linked directly or indirectly to the spacer of the epitope moiety, the spacer may sufficiently space the radionuclide from the recognition sequence so as to not affect the binding of the epitope moiety by the antigen binding domain of the immune cell receptor.

In some embodiments, the spacer comprises at least 1, at least 2, at least 3, at least 4, at least 5, at least 6, at least 7, at least 8, at least 9, or at least 10 amino acids. In some embodiments, each spacer independently comprises no more than 20, no more than 19, no more than 18, no more than 17, no more than 16, no more than 15, no more than 14, no more than 13, no more than 12, no more than 11, no more than 10 amino acids, no more than 9, no more than 8, no more than 7, no more than 6, no more than 5, or no more than 4 amino acids. Any minimum and maximum amount can be combined to form a range provided that the range is within 1 to 20 amino acids, for example from 3 to 15 amino acids or from 5 to 11 amino acids or from 2 to 6 amino acids.

In certain examples, the spacer comprises amino acid residues derived from the dysfunctional P2X₇ receptor sequence, whether located N terminal or C terminal to the epitope sequence. For example, in some embodiments, the spacer is selected from the amino acid sequence C terminal to the core E200 epitope sequence of the receptor, such as the sequence TFHKT (SEQ ID NO: 138) and as exemplified in the peptide defined in SEQ ID NO: 9. Alternatively or additionally, the spacer may include amino acid sequence N terminal to the core E200 epitope sequence of the receptor, such as the amino acid sequence DFP (SEQ ID NO: 139), and as exemplified in the peptide of SEQ ID NO: 140.

It will be appreciated that the radiolabelled molecule, or molecule to be radiolabelled in accordance with the invention, may in further embodiments, be in the form of a fusion protein, wherein the fusion protein comprises a first amino acid sequence comprising an epitope of a dysfunctional P2X₇ receptor and a second amino acid sequence. In certain examples, the second amino acid sequence may comprise a “spacer” sequence, as outlined above. Alternatively, the second amino acid sequence may comprise an amino acid sequence of a protein for increasing the solubility or stability of the molecule. As such, the second amino acid sequence may comprise an amino acid sequence derived from an immunoglobulin, a serum albumin, or other protein such as transferrin, a carboxy-terminal peptide of chorionic gonadotropin (CG) p chain, a non-exact repeat peptide sequence, a polypeptide sequence composed of proline-alanine-serine polymer, an elastin-like peptide (ELP) repeat sequence), a homopolymer of glycine residues or a gelatin-like protein).

In certain examples, the second amino acid sequence may comprise an amino acid sequence from an immunoglobulin, such as an Fc region (eg comprising a CH₂ and/or CH₃ region) or variant thereof. Accordingly, the radiolabelled molecule, or molecule to be radiolabelled may be an Fc-fusion protein comprised of an amino acid sequence of epitope of a dysfunctional P2X₇ receptor, joined to an Fc region of an antibody.

In any embodiment, the amino acid sequence of the epitope of a dysfunctional P2X₇ receptor may be fused via its C terminal region to the N terminal region of an Fc region of an antibody, or variant thereof. In any embodiment, the amino acid sequence of epitope of a dysfunctional P2X₇ receptor may be fused via its N terminal region to the C terminal region of an Fc region of an antibody, or variant thereof.

Preferably, the Fc region of the fusion protein comprises two heavy chain fragments, more preferably the CH₂ and CH₃ domains of said heavy chain.

The Fc region may comprise one or more amino acid sequence modifications compared to naturally occurring Fc sequences. The Fc region may comprise one or more amino acid substitutions, such as substitution of one or more cysteine residues, so as to prevent dimerisation of the molecule to identical molecules. It will be appreciated that any amino acid substitution which prevents dimerisation of the Fc regions may be employed. As such, in vivo, the Fc fusion proteins described herein may be monomeric proteins.

The Fc region of the fusion protein may therefore comprise one or more amino acid substitutions compared to naturally occurring Fc sequences, which prevent or reduce the ability of the Fc region to homodimerise. Preferably, the amino acid substitutions comprise one or more substitutions of the cysteine residues so as to prevent the formation of disulphide bonds between Fc molecules. The cysteine residues of the Fc region may be substituted to any other amino acid residue, optionally to glycine, serine, alanine, lysine and glutamic acid, preferably glycine or serine.

The cysteine residues for substitution are preferably one or more of the cysteine residues located in the region of the Fc region which corresponds to the hinge region of an immunoglobulin. Examples of the IgG1 hinge regions, and variations thereof including cysteine to serine substitutions are provided herein in Table 3. The hinge region of an immunoglobulin (eg of IgG1) comprises three cysteine residues (which are number C₂₂₀, C226 and C229 according to EU numbering. Accordingly, in any embodiment, at least one, at least two, or all three of the cysteine residues in the immunoglobulin hinge region are substituted. Preferably, at least two or all three of the cysteine residues are substituted. More preferably, all cysteine residues in the Fc region, such as the hinge region, are substituted. In particularly preferred embodiments, at least one of C226 and C229 are substituted, preferably wherein both C226 and C229 are substituted.

Accordingly, in preferred embodiments, the fusion protein comprises a hinge region for linking the a dysfunctional P2X₇ receptor epitope moiety and Fc region of an antibody, wherein the hinge region comprises an amino acid sequence that corresponds to any of the sequences set forth in SEQ ID NOs: 76 to 113, or 136 to 137 or 141 or 142.

In further embodiments, the fusion protein region may comprise an Fc region corresponding to an Fc “hole” or “knob” for use in a “knob-in-hole” heterodimer. The use of such Fc sequences is known in the art and provides for an asymmetric heterodimeric molecule comprising a fusion protein with a single copy of the epitope moiety as described herein and Fc region, bound to a further Fc region that does not comprise the epitope moiety.

The skilled person will be familiar with technology and Fc sequences for enabling the formation of so-called monomeric fusion proteins, including although not limited to the use of the “knobs-into-holes” IgG1 format (Ridgway et al., (1996), Protein Eng, 9: 617-621). Such approaches in the context of the present invention, enable expression and purification of a heterodimeric fusion protein with only one copy of the peptide epitope (eg an epitope moiety derived from the E200 epitope as herein described), per molecule. Examples of the “knob-into-hole” Fc pairing are provided herein in SEQ ID NOs: 157 and 159 (knob and hole, respectively), 158 and 159, respectively, 160 and 162 (hole and knob, respectively) and 161 and 162, respectively. Accordingly, in any embodiment, the present invention provides a fusion protein comprising the amino acid sequence of any of SEQ ID NOs: 2 to 69 and 122, linked to an Fc region as defined in SEQ ID NO: 160 or 162, wherein the fusion protein is capable of forming a heterodimer with an Fc region that does not comprise an E200 peptide moiety.

As such, a fusion protein of the invention is preferably one that is capable of forming a heterodimeric molecule that comprises a single E200-containing amino acid sequence. (In other words, the Fc portion of the fusion protein may form a heterodimer with an Fc region of an antibody that does not comprise an E200 peptide fused thereto).

In further embodiments, the Fc region may comprise one or more substitutions for ablating or reducing effector function, such as to reduce binding and activation via the FcR as further described below.

The term “Fc region” herein is used to define a C-terminal region of an immunoglobulin heavy chain that contains at least a portion of the constant region. In other words, the Fc region contains two heavy chain fragments comprising the C_{H}2 and C_{H}3 domains of an antibody. In the context of the present invention, the Fc region comprises two heavy chain fragments, preferably the CH₂ and CH₃ domains of said heavy chain. The two heavy chain fragments are held together by two or more disulfide bonds and by hydrophobic interactions of the C_{H}3 domains. The heavy chain constant domains that correspond to the different classes of immunoglobulins are called α, δ, ε, γ, and μ, respectively.

In some aspects, the fusion protein does not exhibit any effector function or any detectable effector function. “Effector functions” or “effector activities” refer to those biological activities attributable to the Fc region of an antibody, which vary with the antibody isotype. Examples of antibody effector functions include: C₁q binding and complement dependent cytotoxicity (CDC); Fc receptor binding; antibody dependent cell-mediated cytotoxicity (ADCC); phagocytosis; down regulation of cell surface receptors (e.g. B cell receptor); and B cell activation. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability.

The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991).

Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96© non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C₁q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006); WO 2013/120929 A1).

Fc regions with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581). For example, an antibody variant may comprise an Fc region with one or more amino acid substitutions which diminish FcγR binding, e.g., substitutions at positions 234 and 235 of the Fc region (EU numbering of residues). For example, the substitutions are L234A and L235A (LALA) (See, e.g., WO 2012/130831). Further, alterations may be made in the Fc region that result in altered (i.e., diminished) C₁q binding and/or Complement Dependent Cytotoxicity (CDC), e.g., as described in U.S. Pat. No. 6,194,551, WO 99/51642, and Idusogie et al. J. Immunol. 164: 4178-4184 (2000) (eg G236R).

Further examples of modified Fc regions include those comprising the “LALALS” (amino acid substitutions L234A/L235A/M428L/N434S as described in Zalevsky et al., (2010) Nat. Biotechnol. 28: 157-159); the LALAPG (L234A/L235A/P329G amino acid substitutions as described in Gunn et al., (2021, Immunity 54: 815).

In any embodiment, the Fc region of the Fc fusion proteins of the invention may comprise at least the “LALA” mutations (L234A and L235A) for reducing binding to FcR. The fusion protein may in addition or alternatively comprise the mutation G346R for abrogating recruitment of complement C1q.

Other Fc modifications for use in the present invention include variants that reduce or ablate binding to FcγRs and/or complement proteins, thereby reducing or ablating Fc-mediated effector functions such as ADCC, ADCP, and CDC. Such variants are also referred to herein as “knockout variants” or “KO variants”. Variants that reduce binding to FcγRs and complement are useful for reducing unwanted interactions mediated by the Fc region. Preferred knockout variants are described in US 2008-0242845 A1, published on Oct. 2, 2008, entitled “Fc Variants with Optimized Properties, expressly incorporated by reference herein. Preferred modifications include but are not limited substitutions, insertions, and deletions at positions 234, 235, 236, 237, 267, 269, 325, and 328, wherein numbering is according to the EU index. Preferred substitutions include but are not limited to 234G, 235E, 235G, 236R, 237K, 267R, 269R, 325L, and 328R, wherein numbering is according to the EU index. A preferred variant comprises 236R/328R. Variants may be used in the context of any IgG isotype or IgG isotype Fc region, including but not limited to human IgG1, IgG2, IgG3, and/or IgG4. Preferred IgG Fc regions for reducing FcγR and complement binding and reducing Fc-mediated effector functions are IgG2 and IgG4 Fc regions. Hybrid isotypes may also be useful, for example hybrid IgG1/IgG2 isotypes as described in U.S. Ser. No. 11/256,060. Other modifications for reducing FcγR and complement interactions include but are not limited to substitutions 297A, 297D, 234A, 235A, 237A, 318A, 228P, 236E, AG236, 265G, 268Q, 297Q, 309L, 330S, 331S, 327Q, 220S, 226S, 229S, 238S, 233P, 234A, and 234V, as well as removal of the glycosylation at position 297 by mutational or enzymatic means or by production in organisms such as bacteria that do not glycosylate proteins. These and other modifications are reviewed in Strohl, 2009, Current Opinion in Biotechnology 20:685-691, incorporated by reference in its entirety.

In some aspects, the Fc region includes mutations to the complement (C₁q) and/or to Fc gamma receptor (FcγR) binding sites. In some aspects, such mutations can render the fusion protein incapable of antibody directed cytotoxicity (ADCC) and complement directed cytotoxicity (CDC).

The Fc region as used in the context of the present invention preferably does not trigger cytotoxicity such as antibody-dependent cellular cytotoxicity (ADCC) or complement dependent cytotoxicity (CDC).

In some aspect, the Fc region may comprise one or more substitutions for reducing affinity for the FcRn, and thereby reduce serum or circulating half-life of the fusion protein. Substitutions for reducing affinity to FcRn are known in the art and are described for example in Ward et al., (2015), Mol. Immunol., 67: 131-141 and Grevys et al., (2015), 194: 5497-5508. Examples of substitutions include substitutions at one or more of Ile253, His310 and His435, such as 1253A and H310A and H435A.

The term “Fc region” also includes native sequence Fc regions and variant Fc regions. The Fc region may include the carboxyl-terminus of the heavy chain. Antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991. Amino acid sequence variants of the Fc region of an antibody may be contemplated. Amino acid sequence variants of an Fc region of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the Fc region of the antibody. Any combination of deletion, insertion, and substitution can be made to arrive at the final construct, provided that the final construct possesses the desired characteristics, e.g., inducing or supporting an anti-inflammatory response.

The Fc region of the antibody may be an Fc region of any of the classes of antibody, such as IgA, IgD, IgE, IgG, and IgM. The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂.

Accordingly, as used in the context of the present invention, the antibody may be an Fc region of an IgG. For example, the Fc region of the antibody may be an Fc region of an IgG1, an IgG2, an IgG2b, an IgG3 or an IgG4. In some aspects, the fusion protein of the present invention comprises an IgG of an Fc region of an antibody. In the context of the present invention, the Fc region of the antibody is an Fc region of an IgG, preferably IgG1.

The dysfunctional P2X₇ receptor epitope and the Fc region amino acid sequences may be joined or fused directly or via a linker sequence. The linker sequence may be a spacer sequence as herein defined or as exemplified in Table 1 or 3. Alternatively, the linker sequence may be any amino acid based linker sequence commonly in use in the field.

A linker is usually a peptide having a length of up to 20 amino acids although may be up to 50 amino acids in length. The term “linked to” or “fused to” refers to a covalent bond, e.g., a peptide bond, formed between two moieties. Accordingly, in the context of the present invention the linker may have a length of 1, 2, 3, 4, 5, 6, 7, 8, 9, 10, 11, 12, 13, 14, 15, 16, 17, 18, 19, 20, 21 or 22 or more amino acids. For example, the herein provided fusion protein may comprise a linker between the epitope of a dysfunctional P2X₇ receptor and the Fc region of the antibody, such as between the N-terminus of the Fc regions and the C-terminus of dysfunctional P2X₇ receptor epitope. As another example, the herein provided fusion protein may comprise a linker between the dysfunctional P2X₇ receptor epitope and the Fc region of the antibody, such as between the C-terminus of the Fc regions and the N-terminus of the dysfunctional P2X₇ receptor epitope moiety. Particularly, the dysfunctional P2X₇ receptor epitope moiety may be fused via a linker at the C-terminus to the N-terminus of the Fc region. Such linkers have the advantage that they can make it more likely that the different polypeptides of the fusion protein fold independently and behave as expected. Thus, in the context of the present invention the dysfunctional P2X₇ receptor epitope moiety and the Fc region of an antibody may be comprised in a single-chain multi-functional polypeptide.

In some aspects, the fusion protein of the present invention includes a peptide linker. In some aspects, the peptide linker links a dysfunctional P2X₇ receptor epitope moiety with an Fc region of an antibody. In some aspects, the peptide linker can include the amino acid sequence Gly-Gly-Ser (GGS), Gly-Gly-Gly-Ser (GGGS) or Gly-Gly-Gly-Gly-Ser (GGGGS). In some aspects, the peptide linker can include the amino acid sequence GGGGS (a linker of 6 amino acids in length) or even longer. The linker may a series of repeating glycine and serine residues (GS) of different lengths, i.e., (GS)n where n is any number from 1 to 15 or more. For example, the linker may be (GS)3 (i.e., GSGSGS) or longer (GS)11 or longer. It will be appreciated that n can be any number including 1, 2, 3, 4, 5, 6, 7, 8, 9, 10, 11 or more. Fusion proteins having linkers of such length are included within the scope of the present invention. Preferably n is no more than 3 (ie such that when n equals 3 the linker is GSGSGS).

In further embodiments, the linker may comprise inclusion of an amino acid that provides rigidity, such as lysine. For example, in certain embodiments, the linker region may also comprise the sequence GSGK.

The peptide linker may consist of a series of repeats of Thr-Pro (TP) comprising one or more additional amino acids N and C terminal to the repeat sequence. For example, the linker may comprise or consist of the sequence GTPTPTPTPTGEF (also known as the TP5 linker). In further aspects, the linker may be a short and/or alpha-helical rigid linker (e.g. A(EAAAK)3A, PAPAP or a dipeptide such as LE or CC).

In further embodiments, as an alternative or in addition to a glycine-serine-based linker region as described above, the fusion protein may comprise a dysfunctional P2X₇ receptor epitope moiety, linked to an Fc region of an antibody, via a hinge region.

The linking between the dysfunctional P2X₇ receptor epitope moiety and the Fc region may comprise a combination of hinge region and linker regions.

Examples of suitable hinge regions include hinge regions derived from immunoglobulins. The hinge region may be derived from an IgG1, IgG2, IgG3 or IgG4, and may comprise one or more amino acid substitutions, (for example to prevent or reduce the likelihood of disulphide bridge formation). Alternative hinge sequences may be derived from alternative immunoglobulin domains, CD8A, CD8B, CD4 or CD28, TRAC, TRBC, TRGC, TRDC.

Table 3 below provides non-limiting examples of suitable hinge regions for use in joining the dysfunctional P2X₇ receptor epitope moiety and Fc regions, in the molecules of the invention.

It will be appreciated that the dysfunctional P2X₇ receptor epitope moiety may be joined to the Fc regions by more than one linker and/or more than one hinge region. For example, the fusion protein may comprise (N to C terminus), the dysfunctional P2X₇ receptor epitope moiety, conjugated directly to the Fc region. Alternatively, the fusion protein may comprise the dysfunctional P2X₇ receptor epitope moiety, followed by a linker region, then the Fc region. Further still, the fusion protein may comprise the dysfunctional P2X₇ receptor epitope moiety, followed by a linker region, then a hinge region, and then the Fc region. In a further embodiment still, the fusion protein may comprise the dysfunctional P2X₇ receptor epitope moiety, followed by a linker region, then a hinge region, a further linker region, then the Fc region. Of course, the skilled person will appreciate that the alternative configuration is possible (ie wherein the dysfunctional P2X₇ receptor epitope moiety is joined to the C terminus of the Fc region, via one or more linker and/or hinge regions.

TABLE 3
further exemplary linker/hinge region sequences

| Amino acid sequence | SEQ ID NO: | Description |
|---|---|---|
| EPKSSDKTHTSPPSP | 76 | Sequences derived from IgG1 (ie |
| EPKSSDKTHTSPP | 77 | modified IgG1 hinge regions) |
| EPKSSDKTHTSPPP | 78 | |
| EPKSSDKTHT | 79 | |
| DKTHTSPPSP | 80 | |
| DKTHTSPP | 81 | |
| EPKSDKTHTPPP | 82 | |
| EPKSDKTHTSPPSP | 83 | |
| EPKSGDKTHTGPPGP | 84 | |
| EPKSGDKTHTGPP | 85 | |
| EPKSGDKTHTGPPP | 86 | |
| EPKSGDKTHT | 87 | |
| DKTHTGPPGP | 88 | |
| DKTHTGPP | 89 | |
| EPKSDKTHTGPPGP | 90 | |
| EPKSGDKTHTSPPSP | 91 | |
| EPKSGDKTHTGPPSP | 92 | |
| EPKSSDKTHTGPPGP | 93 | |
| EPKSSDKTHTGPPSP | 94 | |
| EPKSSDKTHTGPP | 95 | |
| EPKSGDKTHTSPP | 96 | |
| EPKSTDKTHTTPPTP | 97 | |
| EPKSTDKTHTTPP | 98 | |
| EPKSTDKTHTTPPP | 99 | |
| EPKSTDKTHT | 100 | |
| DKTHTTPPTP | 101 | |
| DKTHTTPP | 102 | |
| EPKSDKTHTTPPTP | 103 | |
| EPKSSDKTHTTPPTP | 104 | |
| EPKSSDKTHTSPPTP | 105 | |
| EPKSADKTHTLPPMP | 106 | |
| EPKSVDKTHTLPPIP | 107 | |
| EPKSLDKTHTAPPAP | 108 | |
| EPKSVDKTHTAPP | 109 | |
| EPKSIDKTHTLPP | 110 | |
| DKTHTAPPLP | 111 | |
| DKTHTVPPLP | 112 | |
| EPKSSDKTHTSP | 113 | |
| EPKSCDKTHTCPPCP | 114 | Wild type IgG1 |
| ERKCCVECPPCP | 115 | Mutated IgG2 |
| ERKXXVEXPPXP | 116 | Mutated IgG2 |
| ERKXXVEXPP | 117 | Mutated IgG2 |
| VEXPPXP | 118 | Mutated IgG2 |
| ELKTPLGDTTHTCPRQP | 119 | Wild-type IgG3 |
| ELKTPLGDTTHTXPRXP | 120 | Mutated IgG3 |
| ELKTPLGDTTHTXPR | 121 | Mutated IgG3 |
| ELKTPLGDTTHT | 123 | Mutated IgG3 |
| EPKSCDTPPPCPRCP | 124 | Wild-type IgG3 |
| EPKSXDTPPPXPRXP | 125 | Mutated IgG3 |
| EPKSXDTPPPXPR | 126 | Mutated IgG3 |
| EPKSXDTPPP | 127 | Mutated IgG3 |
| DTPPPXPRXP | 128 | Mutated IgG3 |
| ESKYGPPCPSCP | 129 | Wild-type IgG4 |
| EXKYGPPCPXCP | 130 | Mutated IgG4 |
| EXKYGPPCP | 131 | Mutated IgG4 |
| KYGPPCPXCP | 132 | Mutated IgG4 |
| EPKSCDKTHTCP | 133 | Wild type IgG1 (v2) |
| EPKSCDTPPPCP | 134 | Wild type IgG3 (v2) |
| ESKYGPPSPSSP | 135 | Mutated IgG4 |
| ERKSSVESPPSP | 136 | Mutated IgG2 |
| EPKSSDTPPPSP | 137 | Mutated IgG3 |

In certain embodiments, the dysfunctional P2X₇ receptor epitope moiety is directly fused to the Fc region of an antibody, such that there is no linker between the two regions of the fusion protein.

The radionuclide may be any radionuclide suitable for use in nuclear medicine, such as nuclear medicine tomographic imaging. The radionuclide may allow the radiolabelled compound of the invention to be detected, for example by a radionuclide scan. In some embodiments, the radionuclide is a positron-emitting radioisotope, which may be detected by positron emission tomography (PET). In some embodiments, the radionuclide is a gamma-emitting isotope, which may be detected by single-photon emission computed tomography (SPECT).

The radionuclide may be linked to the radiolabelled compound of the invention by a covalent bond or a non-covalent bond (eg by co-ordination). In some embodiments, the radionuclide is selected from carbon-11 (¹¹C), fluorine-18 (¹⁸F), scandium-44 (⁴⁴Sc), copper-62, -64 and -67 (⁶²Cu, ⁶⁴Cu, ⁶⁷Cu), gallium-67 and -68 (⁶⁷Ga, ⁶⁸Ga), yttrium-86 and -90 (⁸⁶Y, ⁹⁰Y), zirconium-89 (⁸⁹Zr), niobium-90 (⁹⁰Nb), technetium-94 and -99 (⁹⁴mTc, ⁹⁹mTc), indium-111 (¹¹¹In), iodine-123, -124, -125 and -131 (¹²³I, ¹²⁴I, ¹²⁵I, ¹³¹I), lutetium-177 (¹⁷⁷Lu) and bismuth-123 (²¹³Bi). In some embodiments, the radionuclide is selected from a radioisotope of C, F, Sc, Cu, Ga, Y, Zr, Nb, Tc, In, I, Lu and Bi.

The radionuclide may be directly linked to the epitope moiety, for example linked to an amino acid sidechain. In the case that the epitope moiety comprises a spacer, the radionuclide may be directly linked to an amino acid sidechain of the spacer, which may sufficiently space the radionuclide from the recognition sequence so as to not affect the binding of the epitope moiety. In embodiments where the radionuclide is directly linked to the epitope moiety, the radionuclide may be selected from ¹¹C, ¹⁸F and ^{99m}Tc. Examples of amino acids directly linked to a radionuclide include fluorine-18 labelled Tyrosine (¹⁸F-Tyr) and technetium-99 labelled histidine (^{99m}Tc-His).

The radionuclide may alternatively be indirectly linked to the epitope moiety, for example the radionuclide may be contained in a radiolabelled moiety that is conjugated to the epitope moiety. In some embodiments, the radiolabelled moiety is conjugated to an amino acid sidechain of the epitope moiety. In some embodiments, the radiolabelled moiety is conjugated to the N-terminus or the C-terminus of the epitope moiety.

The radionuclide may be linked to the radiolabelled moiety by a covalent bond. Accordingly, in some embodiments, the radiolabelled moiety comprises a covalently bound radionuclide. In these embodiments, the radionuclide may be selected from ¹¹C, ¹⁸F, ¹²³I, ¹²⁴I, ¹²⁵I, and ¹³¹I.

The radionuclide may alternatively be linked to the radiolabelled moiety by a non-covalent bond (eg by co-ordination). Accordingly, in some embodiments, the radiolabelled moiety comprises a chelator moiety that is capable of chelating a radionuclide, wherein a radionuclide is complexed with the chelator moiety. In these embodiments, the radionuclide may be selected from ⁴⁴Sc, ⁶²Cu, ⁶⁴Cu, ⁶⁷Cu, ⁶⁷Ga, ⁶⁸Ga, ⁸⁶Y, ⁹⁰Y, ⁸⁹Zr, ⁹⁰Nb, ⁹⁴mTc, ⁹⁹mTc, ¹¹¹In, ¹⁷⁷Lu and ²¹³Bi.

The chelator moiety may be any suitable chelator capable of chelating a radionuclide. In some embodiments, the chelator moiety is selected from TMT (6,6″-bis[N, N″,N′″-tetra(carboxymethyl)aminomethyl)-4′-(3-amino-4-methoxyphenyl)-2,2′:6′,2″-terpyridine), DOTA (1,4,7,10-tetraazacyclododecane-NN′,N″(N″-tetraacetic acid, also known as tetraxetan), TCMC (the tetra-primary amide of DOTA), DO3A (1,4,7,10-tetraazacyclododecane-1,4,7-tris(acetic acid)-10-(2-thioethyl)acetamide), CB-DO2A (4,10-bis(carboxymethyl)-1,4,7,10-tetraazabicyclo[5.5.2]tetradecan), NOTA (1,4,7-triazacyclononane-triacetic acid), NETA ({4-[2-(bis-carboxymethyl-aminoethyl]-7-carboxymethyl-[1,4,7]triazonan-1-yl}), diamsar (3,6,10,13,16,19-hexaazabicyclo[6.6.6]eicosane-1,8-diamine), DTPA (pentetic acid or diethylenetriaminepentaacetic acid), CHX-A″-DTPA ([(R)-2-amino-3-(4-isothiocyanatophenyl)propyl]-trans-(S,S)-cyclohexane-1,2-diamine-pentaacetic acid), TETA (1,4,8,11-tetraazacyclotetradecane-1,4,8), 11-tetraacetic acid, Te2A (4,11-bis(carboxymethyl)-1,4,8,11-tetraazabicyclo[6.6.2]hexadecane), HBED (N,N′-bis(2-hydroxybenzyl)ethylenediamine-N,N′-diacetic acid), 5HBED (3,3′-((ethane-1,2-diylbis((carboxymethyl)azanediyl))bis(methylene))bis(4-hydroxybenzenesulfinate)), HYBIC (6-hydrazinonicotinic acid), DFO (desferrioxamine), DFOsq (DFO-squaramide) and HOPO (3,4,3-(LI-1,2-HOPO), or other chelating agent as described herein.

The chelator moiety may be directly conjugated to the epitope moiety, or indirectly conjugated to the epitope moiety by a linker, which may comprise a peptide or a chemical group. The linker may any suitable linker known in the art, provided that the presence of the linker does not substantially affect the ability of the chelator moiety to complex a radionuclide and/or affect the ability of the epitope moiety to bind to an immune cell. The chelator moiety may be conjugated to the epitope moiety or the linker (if present) by any suitable means. By way of non-limiting example, in the case that the chelator moiety is DOTA, the DOTA may be conjugated to the epitope moiety or a linker through at least one of the carboxylic acid groups of DOTA, for example by forming an amide or ester bond with a suitable functionality (eg amine or hydroxyl group) on the epitope moiety or linker. The DOTA may alternatively be conjugated to the epitope moiety or a linker through at least one of the carbon atoms in the tetraazacyclododecane ring and/or through at least one of the methylene groups of at least one of the four carboxylic acid groups of DOTA.

In any embodiment, the radiolabelled moiety may be further conjugated to a further dysfunctional P2X₇ receptor epitope moiety that is recognised or capable of being bound by a receptor expressed on an immune cell, wherein the receptor comprises an antigen recognition domain for binding a dysfunctional P2X₇ receptor and a signalling domain. That is, the radiolabelled compound of the invention may comprise one or more dysfunctional P2X₇ receptor epitope moieties, as described elsewhere herein. In the case that the radiolabelled compound of the invention comprises two or more dysfunctional P2X₇ receptor epitope moieties, the epitope moieties may comprise or consist of the same sequence, or of different sequences.

### Radiolabel-Precursor Molecules

In molecules labelled with a radionuclide, the radionuclide may be susceptible to decay and may have a relatively short half-life. For this reason, radiolabelled molecules may need to be prepared shortly before their intended use (eg before administering to a subject and detecting in vivo via a radionuclide scan), so that the radiolabelled molecule may be used within the expected lifetime of the radionuclide. Ideally, the radiolabelled molecule is prepared from a precursor by a minimal number of reaction steps, which may allow for efficient preparation of the radiolabelled molecule. By way of non-limiting example, the radiolabelled molecule may be prepared from a radiolabel-precursor compound within about 30 minutes.

Accordingly, the present invention also provides a radiolabel-precursor molecule comprising:
(i) a dysfunctional P2X₇ receptor epitope moiety that is recognised or capable of being bound by an antigen binding domain of a receptor expressed on an immune cell, wherein the receptor is for binding a dysfunctional P2X₇ receptor and comprises a signalling domain; and
(ii) a radionuclide-precursor moiety selected from:
   (A) an atom or functionality capable of being converted to a radionuclide;
   (B) a reactive functionality capable of conjugating to a radiolabelled prosthetic group; or
   (C) a chelator moiety capable of chelating a radionuclide, or a salt or solvate thereof.

The radiolabel-precursor molecules of the invention may be used to prepare a radiolabelled molecule as described herein. Accordingly, the present invention also provides the use of the radiolabel-precursor molecule for preparing the radiolabelled molecule as described herein.

As will be apparent to the skilled practitioner, the epitope moiety will typically be any suitable epitope moiety as defined herein for the radiolabelled molecule. However, rather than being directly or indirectly linked to a radionuclide, the epitope moiety is instead directly or indirectly linked to a moiety (such as any of (A), (B) or (C) as mentioned above), which can be converted to a radionuclide, or can be conjugated or chelated to a radionuclide.

The radiolabel-precursor molecule may comprise a radionuclide-precursor moiety conjugated to the epitope moiety, wherein the radiolabel-precursor moiety comprises one of (A), (B) or (C) above. Accordingly, any of (A), (B) and (C) may be present in the epitope moiety or a radiolabel-precursor moiety conjugated to the epitope moiety.

Any suitable atom or functionality capable of being converted to a radionuclide known in the art may be used. The atom or functionality may be converted to a radionuclide, for example, by substitution, addition or exchange with a compound comprising the radionuclide. By way of non-limiting example, the atom or functionality may be any suitable leaving group capable of being substituted by a radionuclide (eg ¹⁸F). In the case that the atom or functionality is present in the epitope moiety, the atom or functionality may be, for example, the hydroxyl group of a tyrosine side chain, which may be substituted by ¹⁸F to provide ¹⁸F-Tyr. In the case that the atom or functionality is present in the radiolabel-precursor moiety, the atom or functionality may be, for example, any suitable leaving group as described in Krishnan et al (¹⁸F-Labeling of Sensitive Biomolecules for Positron Emission Tomography, Chemistry. 2017 Nov. 7; 23(62): 15553-15577). By way of further non-limiting example, the atom or functionality may be an iodine atom capable of being exchanged with ¹²⁵I or other iodine isotope.

Any suitable reactive functionality capable of conjugating a radiolabelled prosthetic group known in the art may be used. It will be appreciated that the reactive functionality will be capable forming a covalent bond with a complementary reactive functionality present on the radiolabelled prosthetic group. In the case that the reactive functionality is present in the epitope moiety, the reactive functionality may be, for example, a reactive functionality of an amino acid sidechain (eg a cysteine thiol group). In the case that the reactive group is present in a radiolabel-precursor moiety, the reactive group may be conjugated to the epitope moiety by any suitable linker, which may comprise a peptide or a chemical group.

In some embodiments, the reactive functionality is an amino group, which can form an amide bond to a radiolabelled prosthetic group comprising a carboxylic acid group. In some embodiments, the reactive functionality is a carboxylic acid group, which can form an amide bond to a radiolabelled comprising an amine group. In some embodiments, the reactive functionality is a hydroxyl group, which can form an ester bond to a radiolabelled prosthetic group comprising a carboxylic acid group. In some embodiments, the reactive functionality is a carboxylic acid group, which can form an ester bond to a radiolabelled prosthetic group comprising a hydroxyl group. In some embodiments, the reactive functionality comprises a leaving group (such as, but not limited, to a halogen, tosylate, mesylate, triflate, and the like), which can be coupled through nucleophilic displacement to a radiolabelled prosthetic group comprising a nucleophilic group (such as, but not limited to, a thiol, hydroxyl, amine, or carboxylic acid). In some embodiments, the reactive functionality comprises a nucleophilic group (such as, but not limited to, a thiol, hydroxyl, amine, or carboxylic acid), which can be coupled through nucleophilic displacement to a radiolabelled prosthetic group comprising a leaving group (such as, but not limited to, a halogen, tosylate, mesylate, triflate, and the like). In some embodiments, the reactive functionality comprises a group that is represented with an open valency (such as the generic alkyl group R—CH₂—), and that group can be linked through a single covalent bond to a radiolabelled prosthetic group. This list is not exhaustive and is intended to be illustrative only. Any other reactions and reagents known to promote intermolecular coupling are incorporated herein. Any of these compounds and the corresponding conjugates are contemplated within the invention. By way of further non-limiting example, the reactive functionality (or complementary reactive functionality) may be capable of forming a covalent bond with a nitrogen-containing functional group (eg amine) or sulphur-containing functional group (eg thiol), such as —C(═O)CH═CH₂, —S(═O)CH═CH₂, —S(═O)₂CH═CH₂, —C(═O)CH═CH—CH₂NR₂, —S(═O)CH═CH—CH₂NR₂, —S(═O)₂CH═CH—CH₂NR₂, C(═O)C═CH, S(═O)C═CH, S(═O)₂C═CH, α,β-unsaturated ketones, α,β-unsaturated esters, α,β-unsaturated amides, α,β-unsaturated sulphones, α,β-unsaturated sulphoxides, α,β-unsaturated sulphonamides, propargyl ketones, propargyl esters, propargyl amides, propargyl sulphones, propargyl sulphoxides, propargyl sulphonamides, maleimides, α-chloro amides, disulphides, 5-fluoro-2,4-dinitrobenzene, and so forth, or any other amine- or thiol-modifying functionality known in the art, including those described in Krishnan et al (¹⁸F-Labeling of Sensitive Biomolecules for Positron Emission Tomography, Chemistry. 2017 Nov. 7; 23(62): 15553-15577). It will be appreciated that the reactive functionality present in the radiolabel-precursor compound and the complementary reactive functionality present in the radiolabelled prosthetic group may be reversed.

In some embodiments, the reactive functionality is capable of reacting with a radiolabelled prosthetic group via click chemistry, for example as described in Krishnan et al (¹⁸F-Labeling of Sensitive Biomolecules for Positron Emission Tomography, Chemistry. 2017 Nov. 7; 23(62): 15553-15577). In some embodiments, the reactive functionality is an alkyne, which can react with a radiolabelled prosthetic group containing an azide group via click chemistry. In some embodiments, the reactive functionality is an azide, which can react with a radiolabelled prosthetic group containing an alkyne group via click chemistry. Suitable alkynes include strained alkynes, for example dibenzocyclooctyne (DBCO), bicyclononyne (BCN), monofluorooctyne (MOFO), and difluorocyclooctyne (DIFO). In some embodiments, the reactive functionality is a tetrazine, which can react with a radiolabelled prosthetic group containing an alkene via click chemistry. In some embodiments, the reactive functionality is an alkene, which can react with a radiolabelled prosthetic group comprising a tetrazine via click chemistry. Suitable alkenes include strained alkenes, such as trans-cyclooctene (TCO), cyclooctyne and norbornene.

The radiolabelled prosthetic group may be any suitable radiolabelled prosthetic group capable of conjugating to the epitope moiety by reacting with the reactive functionality of the epitope moiety. Non-limiting examples of radiolabelled prosthetic groups comprising a covalently bound radionuclide include those described in Krishnan et al (¹⁸F-Labeling of Sensitive Biomolecules for Positron Emission Tomography, Chemistry. 2017 Nov. 7; 23(62): 15553-15577). In the case that the radionuclide is linked to the radiolabelled prosthetic group by a non-covalent bond, the radiolabelled moiety may a chelator moiety that is capable of chelating a radionuclide, wherein a radionuclide is complexed with the chelator moiety. In this case, the chelator moiety may be the same chelator moiety as defined for the radiolabelled compound described herein.

The radiolabelled prosthetic group may be further conjugated (or capable of being further conjugated) to a further dysfunctional P2X₇ receptor epitope moiety that is recognised or capable of being bound by a receptor expressed on an immune cell, wherein the receptor comprises an antigen recognition domain for binding a dysfunctional P2X₇ receptor and a signalling domain.

Any suitable chelator moiety capable of chelating a radionuclide may be used. The chelator moiety may be present in the epitope moiety. In this case, the chelator moiety may be, for example, a histidine residue capable of chelating a radionuclide (eg ^{99m}Tc, to provide ^{99m}Tc-His). Alternatively, the chelator moiety may be present in a radiolabel-precursor conjugated to the epitope moiety. In this case, the chelator moiety may be the same chelator moiety as defined for the radiolabelled compound described herein. Further, the chelator moiety may be conjugated to the epitope moiety by any suitable linker as described herein.

### Dysfunctional P2X₇ Receptor Epitope Moiety

The dysfunctional P2X₇ receptor epitope moiety may be provided in the form of a dysfunctional P2X₇ receptor, or a fragment of a dysfunctional P2X₇ receptor, that has at least one of the three ATP binding sites that are formed at the interface between adjacent correctly packed monomers that are unable to bind ATP. Such receptors are unable to extend the opening of the non-selective calcium channels to apoptotic pores.

In accordance with the present invention, the dysfunctional P2X₇ receptor epitope moiety is typically in the form of a peptide fragment of a dysfunctional P2X₇ receptor. Accordingly, in particularly preferred embodiments, the radiolabelled molecules of the invention comprise:
(i) a peptide that is recognised or capable of being bound by an antigen recognition domain of a receptor expressed on an immune cell, wherein the receptor is for binding a dysfunctional P2X₇ receptor and comprises a signalling domain; and
(ii) a radionuclide that is directly or indirectly linked to the epitope moiety,
or a salt or solvate thereof.

Further, the present invention provides a radiolabel-precursor molecule comprising:
(i) a peptide that is recognised or capable of being bound by an antigen binding domain of a receptor expressed on an immune cell, wherein the receptor is for binding a dysfunctional P2X₇ receptor and comprises a signalling domain; and
(ii) a radionuclide-precursor moiety selected from:
   (A) an atom or functionality capable of being converted to a radionuclide;
   (B) a reactive functionality capable of conjugating to a radiolabelled prosthetic group; or
   (C) a chelator moiety capable of chelating a radionuclide,
or a salt or solvate thereof.

Typically, the peptide comprises an epitope that is not found or not available for binding on a functional P2X₇ receptor.

In some embodiments, the peptide comprises the proline at amino acid position 210 of the dysfunctional P2X₇ receptor. In some embodiments, the peptide comprises one or more amino acid residues spanning from glycine at amino acid position 200 to cysteine at amino acid position 216, inclusive, of the dysfunctional P2X₇ receptor.

A range of peptide fragments of a dysfunctional P2X₇ receptor are known and discussed in PCT/AU2002/000061 (and in corresponding publications WO 2002/057306 and U.S. Pat. Nos. 7,326,415, 7,888,473, 7,531,171, 8,080,635, 8,399,617, 8,709,425, 9,663,584, or U.S. Pat. No. 10,450,380), PCT/AU2008/001364 (and in corresponding publications WO 2009/033233 and U.S. Pat. Nos. 8,440,186, 9,181,320, 9,944,701 or 10,597,45) and PCT/AU2009/000869 (and in corresponding publications WO 2010/000041 and U.S. Pat. Nos. 8,597,643, 9,328,155 or 10,238,716) the contents of all of which are incorporated in entirety. Exemplary peptides within these specifications which include epitopes contemplated for use in this invention are described below.

| PCT publication | Peptide sequence |
|---|---|
| WO 2002/057306 | GHNYTTRNILPGLNIT |
| | (SEQ ID NO: 3) |
| WO 2002/057306 | GHNYTTRNILPGLNITC |
| | (SEQ ID NO: 2) |
| | (also referred to |
| | herein as the “E200” epitope) |
| WO 2009/033233 | KYYKENNVEKRTLIKVF |
| | (SEQ ID NO: 4) |
| | (also referred to |
| | herein as the “E300” epitope) |
| WO 2010/000041 | GHNYTTRNILPGAGAKYYKENNVEK |
| | (SEQ ID NO: 6) (also |
| | referred to herein as the |
| | “E200/E300” or “composite” epitope) |

Non-limiting examples of variations of the E200 peptide sequence (including with N and/or C terminal extensions, and various linker, hinge or spacer regions) are provided in Table 1.

The amino acid sequences of any one of SEQ ID NOs: 2 to 7 may comprise a portion of the epitope moiety that is recognised or capable of being bound by a receptor expressed on an immune cell (also referred to herein as the “recognition sequence” of the epitope moiety).

In some embodiments, the epitope moiety comprises or consists of an amino acid sequence selected from any of the peptide sequences listed in Table 1 above.

In the case of those peptides in Table 1 having histidine residues, the radionuclide could be conjugated to the epitope moiety via the two histidine residues. In the case of sequences having cysteine residues, conjugation could be via labelling of the cysteine residues using F18 compounds described herein, such as N—[N—(S)-1,3-dicarboxypropyl]carbamol]-4-[¹⁸F]fluorobenzyl-L-cysteine (¹⁸F-DCFBC). In the case of sequences having lysine residues, coupling of the radionuclide may be via the lysine residue.

In some embodiments, the N-terminus of the epitope moiety is a free amine (—NH₂).

In some embodiments, the C-terminus of the epitope moiety is a free acid (—COOH). In some embodiments, the C-terminus is a derivative or analogue of a free acid group, for example an ester (—COOC1-6 alkyl) or a primary or secondary amide (—CONHR4 wherein R4 is selected from H and C₁₋₆ alkyl). Advantageously, having a C-terminus that is a derivative or analogue of a free acid group may improve the biological stability of the peptide compared to the free acid. In some embodiments, the C-terminus is a derivative or analogue of a free acid group that comprises a functional moiety, for example biotin.

### Receptors and Immune Cells Expressing Same

In any embodiment, the receptor that comprises the antigen binding domain for binding to dysfunctional P2X₇ receptor, and signalling domain is preferably a chimeric antigen receptor (CAR) or a variant thereof. The receptor may also be a modified TCR.

In general, a CAR, variant thereof, or TCR, may comprise an extracellular domain (extracellular part) comprising the antigen binding domain, a transmembrane domain and an intracellular signaling domain. The extracellular domain may be linked to the transmembrane domain by a linker. The extracellular domain may also comprise a signal peptide. Preferably, the extracellular part of the CAR, variant thereof, or TCR comprises an nfP2X₇ binding domain that recognises the E200 (or E300 or E200-300 composite) epitope as disclosed herein.

Typically, the antigen-recognition domain includes a binding polypeptide that includes amino acid sequence homology to one or more complementarity determining regions (CDRs) of an antibody that binds to a dysfunctional P2X₇ receptor. In any embodiment, the binding polypeptide includes amino acid sequence homology to the CDR1, 2 and 3 domains of the VH and/or VL chain of an antibody that binds to a dysfunctional P2X₇ receptor.

In preferred embodiments, the binding polypeptide comprises the amino acid sequence of the CDRs of the VH and/or VL chain of an antibody described in PCT/AU2002/000061 or PCT/AU2002/001204 (or in any one of the corresponding U.S. Pat. Nos. 7,326,415, 7,888,473, 7,531,171, 8,080,635, 8,399,617, 8,709,425, 9,663,584, or U.S. Pat. No. 10,450,380), PCT/AU2007/001540 (or in corresponding U.S. Pat. No. 8,067,550), PCT/AU2007/001541 (or in corresponding US publication US 2010-0036101), PCT/AU2008/001364 (or in any one of the corresponding U.S. Pat. Nos. 8,440,186, 9,181,320, 9,944,701 or U.S. Pat. No. 10,597,451), PCT/AU2008/001365 (or in any one of the corresponding U.S. Pat. No. 8,293,491 or U.S. Pat. No. 8,658,385), PCT/AU2009/000869 (or in any one of the corresponding U.S. Pat. Nos. 8,597,643, 9,328,155 or 10,238,716), PCT/AU2010/001070 (or in any one of the corresponding publications WO/2011/020155, U.S. Pat. Nos. 9,127,059, 9,688,771, or 10,053,508), and PCT/AU2010/001741 (or in any one of the corresponding publications WO 2011/075789 or U.S. Pat. No. 8,835,609) the entire contents of which are hereby incorporated by reference. Preferably the antibody comprises the CDR amino acid sequences of 2-2-1 described in PCT/AU2010/001070 (or in any one of the corresponding U.S. Pat. Nos. 9,127,059, 9,688,771, or 10,053,508) or BPM09 described in PCT/AU2007/001541 (or in corresponding US publication US 2010-0036101) and produced by the hybridoma AB253 deposited with the European Collection of Cultures (ECACC) under Accession no. 06080101.

In further embodiments, the binding polypeptide of the CAR comprises the amino acid sequence of the VH and/or VL chains of an antibody described in PCT/AU2002/000061 or PCT/AU2002/001204 (or in any one of the corresponding U.S. Pat. Nos. 7,326,415, 7,888,473, 7,531,171, 8,080,635, 8,399,617, 8,709,425, 9,663,584, or 10,450,380), PCT/AU2007/001540 (or in corresponding U.S. Pat. No. 8,067,550), PCT/AU2007/001541 (or in corresponding US publication US 2010-0036101), PCT/AU2008/001364 (or in any one of the corresponding U.S. Pat. Nos. 8,440,186, 9,181,320, 9,944,701 or 10,597,451), PCT/AU2008/001365 (or in any one of the corresponding U.S. Pat. No. 8,293,491 or 8,658,385), PCT/AU2009/000869 (or in any one of the corresponding U.S. Pat. Nos. 8,597,643, 9,328,155 or 10,238,716), PCT/AU2010/001070 (or in any one of the corresponding publications WO/2011/020155, U.S. Pat. Nos. 9,127,059, 9,688,771, or 10,053,508), and PCT/AU2010/001741 (or in any one of the corresponding publications WO 2011/075789 or U.S. Pat. No. 8,835,609) the entire contents of which are hereby incorporated by reference. Preferably the antibody comprises the CDR amino acid sequences of 2-2-1 described in PCT/AU2010/001070 (or in any one of the corresponding U.S. Pat. Nos. 9,127,059, 9,688,771, or 10,053,508) or BPM09 described in PCT/AU2007/001541 (or in corresponding US publication US 2010-0036101) and produced by the hybridoma AB253 deposited with the European Collection of Cultures (ECACC) under Accession no. 06080101.

In further embodiments still, the binding polypeptide of the CAR comprises the amino acid sequence of an antibody or fragment thereof described in PCT/AU2002/000061 or PCT/AU2002/001204 (or in any one of the corresponding U.S. Pat. Nos. 7,326,415, 7,888,473, 7,531,171, 8,080,635, 8,399,617, 8,709,425, 9,663,584, or 10,450,380), PCT/AU2007/001540 (or in corresponding U.S. Pat. No. 8,067,550), PCT/AU2007/001541 (or in corresponding US publication US 2010-0036101), PCT/AU2008/001364 (or in any one of the corresponding U.S. Pat. Nos. 8,440,186, 9,181,320, 9,944,701 or 10,597,451), PCT/AU2008/001365 (or in any one of the corresponding U.S. Pat. Nos. 8,293,491 or 8,658,385), PCT/AU2009/000869 (or in any one of the corresponding U.S. Pat. Nos. 8,597,643, 9,328,155 or 10,238,716), PCT/AU2010/001070 (or in any one of the corresponding publications WO/2011/020155, U.S. Pat. Nos. 9,127,059, 9,688,771, or 10,053,508), and PCT/AU2010/001741 (or in any one of the corresponding publications WO 2011/075789 or U.S. Pat. No. 8,835,609) the entire contents of which are hereby incorporated by reference. Preferably the antibody comprises the CDR amino acid sequences of 2-2-1 described in PCT/AU2010/001070 (or in any one of the corresponding U.S. Pat. Nos. 9,127,059, 9,688,771, or 10,053,508) or BPM09 described in PCT/AU2007/001541 (or in corresponding US publication US 2010-0036101) and produced by the hybridoma AB253 deposited with the European Collection of Cultures (ECACC) under Accession no. 06080101.

A “signal peptide” refers to a peptide sequence that directs the transport and localisation of the protein within a cell, e.g. to a certain cell organelle (such as the endoplasmic reticulum) and/or the cell surface.

Generally, an “antigen binding domain” (or antigen recognition domain) refers to the region of the CAR that specifically binds to an antigen (and thereby is able to target a cell containing the antigen). A CAR may comprise one or more antigen binding domains. Generally, the targeting regions on the CAR are extracellular. The antigen binding domain may comprise an antibody or an antibody binding fragment thereof. The antigen binding domain may comprise, for example, full length heavy chain, Fab fragments, single chain Fv (scFv) fragments, divalent single chain antibodies or diabodies. Any molecule that binds specifically to a given antigen such as affibodies or ligand binding domains from naturally occurring receptors may be used as an antigen binding domain. Often the antigen binding domain is a scFv. Normally, in an scFv the variable regions of an immunoglobulin heavy chain and light chain are fused by a flexible linker to form a scFv. Such a linker may be for example the “(G₄/S₁)₃-linker” and variations thereof but the skilled person will appreciate that various linker sequences and formats may be used.

CARs may also comprise a “hinge” region (sometimes called a spacer region or linker region) joining the antigen binding domain to the transmembrane domain. This is typically a hydrophilic region that is between the antigen binding domain and the transmembrane domain. A CAR may comprise an extracellular hinge domain but it is also possible to leave out such a hinge. The hinge region may include for example, Fc fragments of antibodies or fragments thereof, hinge regions of antibodies or fragments thereof, CH2 or CH3 regions of antibodies, accessory proteins, artificial hinge sequences or combinations thereof. One example of a hinge region is the CD8alpha hinge.

The transmembrane domain of the CAR may be derived from any desired natural or synthetic source for such a domain. When the source is natural, the domain may be derived from any membrane-bound or transmembrane protein. The transmembrane domain may be derived for example from CD8alpha or CD28. When the key signalling and antigen recognition modules (domains) are on two (or even more) polypeptides, then the CAR may have two (or more) transmembrane domains. The splitting of key signalling and antigen recognition modules enables small molecule-dependent, titratable and reversible control over CAR cell expression (Wu et al, 2015, Science 350: 293-303) due to small molecule-dependent heterodimerising domains in each polypeptide of the CAR.

The cytoplasmic domain (or the intracellular signaling domain) of the CAR is responsible for activation of at least one of the normal effector functions of the immune cell in which the CAR is expressed. “Effector function” means a specialised function of a cell, e.g. in a T cell an effector function may be cytolytic activity or helper cell activity including the secretion of cytokines. The intracellular signalling domain refers to the part of a protein that transduces the effector function signal and directs the cell expressing the CAR to perform a specialised function. The intracellular signalling domain may include any complete, mutated or truncated part of the intracellular signalling domain of a given protein sufficient to transduce a signal that initiates or blocks immune cell effector functions.

The function of the intracellular domains may be pro- or anti-inflammatory and/or immunomodulatory, or a combination of such.

Prominent examples of intracellular signalling domains for use in the CARs include the cytoplasmic signaling sequences of the T cell receptor (TCR) and co-receptors that initiate signal transduction following antigen receptor engagement.

Generally, T cell activation can be mediated by two distinct classes of cytoplasmic signalling sequences, firstly those that initiate antigen-dependent primary activation through the TCR (primary cytoplasmic signalling sequences) and secondly those that act in an antigen-independent manner to provide a secondary or co-stimulatory signal (secondary cytoplasmic signalling sequences, co-stimulatory signalling domain). Therefore, an intracellular signalling domain of a CAR may comprise one or more primary cytoplasmic signalling domains and/or one or more secondary cytoplasmic signalling domains.

Primary cytoplasmic signalling sequences that act in a stimulatory manner may contain ITAMs (immunoreceptor tyrosine-based activation motifs) signalling motifs.

Examples of ITAM containing primary cytoplasmic signalling sequences often used in CARs are those derived from TCR zeta (CD3 zeta), FcR gamma, FcR beta, CD3 gamma, CD3 delta, CD3 epsilon, CD5, CD22, CD79a, CD79b and CD66d. Most prominent is the sequence derived from CD3 zeta.

The cytoplasmic domain of the CAR may be designed to comprise the CD3-zeta signaling domain by itself or combined with any other desired cytoplasmic domain(s). The cytoplasmic domain of the CAR can comprise a CD3 zeta chain portion and a co-stimulatory signalling region. The co-stimulatory signalling region refers to a part of the CAR comprising the intracellular domain of a co-stimulatory molecule. A co-stimulatory molecule is a cell surface molecule other than an antigen receptor or their ligands that is required for an efficient response of lymphocytes to an antigen. Examples for a co-stimulatory molecule are CD27, CD28, 4-1BB (CD137), OX40, CD30, CD40, PD-1, ICOS, lymphocyte function-associated antigen-1 (LFA-1), CD2, CD7, LIGHT, NKG2C and B7-H3.

In some embodiments, the activation receptor (from which a portion of signalling domain is derived) is the CD3 co-receptor complex or is an Fc receptor.

In some embodiments, the co-stimulatory receptor (from which a portion of signalling domain is derived) is selected from the group consisting of CD27, CD28, CD-30, CD40, DAP10, OX40, 4-1BB (CD137) and ICOS.

In some embodiments, the co-stimulatory receptor (from which a portion of signalling domain is derived) is selected from the group consisting of CD28, OX40 or 4-1BE.

The cytoplasmic signalling sequences within the cytoplasmic signalling part of the CAR may be linked to each other with or without a linker in a random or specified order. A short oligo- or polypeptide linker, which is preferably between 2 and 10 amino acids in length, may form the linkage. A prominent linker is the glycine-serine doublet.

As an example, the cytoplasmic domain may comprise the signalling domain of CD3-zeta and the signalling domain of CD28. In another example the cytoplasmic domain may comprise the signalling domain of CD3-zeta and the signalling domain of CD27. In a further example, the cytoplasmic domain may comprise the signalling domain of CD3-zeta, the signalling domain of CD28, and the signalling domain of CD27.

As aforementioned, either the extracellular part or the transmembrane domain or the cytoplasmic domain of a CAR may also comprise a heterodimerising domain for the aim of splitting key signalling and antigen recognition modules of the CAR.

The CAR which binds to a radiolabeled molecule of an invention, e.g., a CAR comprising an nfP2X₇ E200 binding domain, may be designed to comprise any portion or part of the above-mentioned domains as described herein in any order and/or combination resulting in a functional CAR.

The affinity at which the dysfunctional P2X₇ receptor binding domain of the CAR binds to the nfP2X₇ recognition site E200 of a radiolabeled molecule of the invention can vary, but generally the binding affinity may be in the range of approximately 100 μM, approximately 10 μM, approximately 1 μM, approximately 100 nM, approximately 10 nM, or approximately 1 nM, preferably at least about 10 μM or 1 μM. In preferred embodiments, the binding affinity is at least about 1 nM or at least about 10 nM.

The receptor (such as a CAR, variant thereof, or TCR, or variant thereof) is typically expressed by an immune cell.

The immune cell may be an “engineered cell”, “genetically modified cell”, or “immune effector cell” as described herein. Further, the immune cell may be an immune cell precursor that is capable of differentiating into an immune cell. A cell that is capable of differentiating into an immune cell (e.g. T cell that will express the dysfunctional P2X₇ CAR) may be a stem cell, multi-lineage progenitor cell or induced pluripotent stem.

The immune cell may be a leukocyte, a Peripheral Blood Mononuclear Cell (PBMC), a lymphocyte, a T cell (including a CD4+ T cell or a CD8+ T cell), a natural killer cell, a natural killer T cell, or a γδ T cell.

In any embodiment, the immune cell may be a T cell, wherein optionally said T cell does not express TcRαβ, PD1, CD3 or CD96 (e.g. by way of knocking down or knocking out one of these genes on a genetic level or functional level).

In any embodiment, the immune cell optionally does not express accessory molecules that can be checkpoint, exhaustion or apoptosis-associated signalling receptors as well as ligands such as PD-1, LAG-3, TIGIT, CTLA-4, FAS-L and FAS-R, (e.g. by way of knocking out one of these genes on a genetic level or functional level).

In some embodiments, the genetically modified cell includes two or more different receptors (e.g., two or more CARs, or variants thereof). The CARs may bind to different epitopes on the same target molecule (e.g., different epitopes on dysfunctional P2X₇ receptor). Alternatively, the CARs may bind different target molecules, such that only one of the CARs binds to dysfunctional P2X₇ receptors.

As used herein, the term “different CARs” or “different chimeric antigen receptors” refers to any two or more CARs that have either non-identical antigen-recognition and/or non-identical signalling domains. In one example, “different CARs” includes two CARs with the same antigen-recognition domains (e.g. both CARs may recognise a dysfunctional P2X₇ receptor), but have different signalling domains, such as one CAR having a signalling domain with a portion of an activation receptor and the other CAR having a signalling domain with a portion of an co-stimulatory receptor. As will be understood, at least one of the two or more CARs within this embodiment will have an antigen-recognition domain that recognises the dysfunctional P2X₇ receptor and the other CAR(s) may take any suitable form and may be directed against any suitable antigen.

### Methods of Preparing a Radiolabelled Molecule

The present invention further provides a method of preparing a radiolabelled molecule, comprising:
providing a radiolabel-precursor molecule as defined herein; and
reacting the radiolabel-precursor molecule to provide a radiolabelled molecule as defined herein,
thereby providing a radiolabelled molecule.

The radiolabel-precursor molecule may be suitably reacted to provide the radiolabelled molecule depending on the nature of the radiolabel-precursor moiety.

In some embodiments, the method comprises:
providing a radiolabel-precursor molecule comprising:
(i) a dysfunctional P2X₇ receptor epitope moiety that is recognised or capable of being bound by an antigen binding domain of a receptor expressed on an immune cell, wherein the receptor is for binding a dysfunctional P2X₇ receptor and comprises a signalling domain; and
(ii) an atom or functionality capable of being converted to a radionuclide,
or a salt or solvate thereof, and
converting the atom or functionality to a radionuclide;
thereby providing a radiolabelled molecule.

In some embodiments, the method comprises:
providing a radiolabel-precursor molecule comprising:
(i) a dysfunctional P2X₇ receptor epitope moiety that is recognised or capable of being bound by an antigen binding domain of a receptor expressed on an immune cell, wherein the receptor is for binding a dysfunctional P2X₇ receptor and comprises a signalling domain; and
(ii) a reactive functionality capable of conjugating to a radiolabelled prosthetic group,
or a salt or solvate thereof, and
conjugating the radiolabelled prosthetic group via the reactive functionality;
thereby providing a radiolabelled molecule.

In some embodiments, the method comprises:
providing a radiolabel-precursor molecule comprising:
(i) a dysfunctional P2X₇ receptor epitope moiety that is recognised or capable of being bound by an antigen binding domain of a receptor expressed on an immune cell, wherein the receptor is for binding a dysfunctional P2X₇ receptor and comprises a signalling domain; and
(ii) a chelator moiety capable of chelating a radionuclide, or a salt or solvate thereof, and
chelating a radionuclide to the chelator moiety;
thereby providing a radiolabelled molecule.

In the above methods, the atom or functionality capable of being converted to a radionuclide, the reactive functionality capable of conjugating to a radiolabelled prosthetic group, and the chelator moiety capable of chelating a radionuclide may be present in the epitope moiety or a radiolabel-precursor moiety conjugated to the epitope moiety, as described herein.

The radiolabel-precursor molecule may be suitably prepared and reacted to provide the radiolabelled molecule by methods known in the art, including methods described herein. Suitable methods for obtaining radiolabelled peptide (such as for coupling ⁶⁸Ga to a DOTA-like conjugated peptide) are described for example in Mueller et al., (2011) Nature Protocols, 11: 1057-1066, incorporated herein by reference.

The epitope moiety of the radiolabelled molecules or radiolabel-precursor molecules of the invention may be prepared by known chemical methods, including solid-phase and solution-phase peptide synthesis using Fmoc or Boc protected amino acid residues. The epitope moiety may also be prepared by known recombinant DNA technologies.

The radiolabelling of the peptide may be via one or more histidine residues present in the peptide. Examples of radiolabelling of histidine residues are well known in the art, and are described for example in Ibrahim et al., (2016) Radiochemistry, 58: 521-527, incorporated herein by reference. In such instances, the peptide may comprise a biotin label at the C terminus, or an amide.

In further examples, the tyrosine residue(s) in the peptide could be labelled using standard techniques known to the skilled person.

Further still, radiolabelling could be of the cysteine residues using F18 compounds such as N—[N—(S)-1,3-dicarboxypropyl]carbamol]-4-[¹⁸F]fluorobenzyl-L-cysteine (¹⁸F-DCFBC). In such case, the peptide would preferably comprise the amino acid sequence as shown in any of SEQ ID NOs: 2, 10 or 13 (eg: GHNYTTRNILPGLNITSTFHKTC-amide). Methods for 18F labelling are described in David et al., (2019) RSC Adv. 15: 8638-8649, incorporated herein by reference.

### Applications

The radiolabelled molecule of the invention may be useful for detecting an immune cell expressing a receptor comprising an antigen recognition domain for binding a dysfunctional P2X₇ receptor and a signalling domain. The radiolabelled molecule comprises a dysfunctional P2X₇ receptor epitope moiety that is recognised or capable of being bound by a receptor expressed on said immune cell. The presence of the dysfunctional P2X₇ receptor epitope moiety may therefore allow the molecule of the invention to bind to said immune cell.

Accordingly, the present invention provides the use of a radiolabelled molecule described herein for detecting an immune cell expressing a receptor comprising an antigen recognition domain for binding a dysfunctional P2X₇ receptor and a signalling domain.

The present invention also provides a method of detecting an immune cell expressing a receptor comprising an antigen recognition domain for binding a dysfunctional P2X₇ receptor and a signalling domain in a subject, comprising:
administering a radiolabelled molecule as described herein to a subject who has been administered an immune cell expressing a receptor comprising an antigen recognition domain for binding a dysfunctional P2X₇ receptor; and
detecting the radiolabelled compound in the subject, wherein presence of the radiolabelled compound indicates the presence of the immune cell.

In some embodiments, the radiolabelled molecule is detected by performing a radionuclide scan. The radionuclide scan may be suitably selected depending on the radionuclide present in the radiolabelled molecule. In some embodiments, the radionuclide scan may be a positron emission tomography (PET) scan or a single-photon emission computerized tomography (SPECT) scan.

In some embodiments, the method further comprises imaging the detected radiolabelled molecule.

In some embodiments, the method further comprises allowing the radiolabelled molecule to concentrate at sites in the subject where the immune cell is found, prior to the step of detecting the radiolabelled molecule.

In some embodiments, the method further comprises administering an immune cell expressing a receptor comprising an antigen recognition domain for binding a dysfunctional P2X₇ receptor to the subject, prior to the step of administering the radiolabelled molecule to the subject.

The methods and uses of the radiolabelled molecule described herein may advantageously allow (i) determining whether immune cells expressing a receptor comprising an antigen recognition domain for binding a dysfunctional P2X₇ receptor and a signalling domain are present in a subject, (ii) identifying the location(s) of said immune cells, including determining the distribution a population of said immune cells in a subject and (iii) quantifying the number of said immune cells in a subject or at a particular location/site within the subject. This information may assist with informing the development or adjustment of a treatment regimen using said immune cells.

### Compositions and Formulations

The radiolabelled molecule may be provided in a suitable form or formulated for administration to a subject.

Accordingly, the present invention provides a composition comprising the radiolabelled molecule of the invention, or a salt or solvate thereof.

The composition may be a pharmaceutical composition. In the case of a pharmaceutical composition, the composition may comprise a pharmaceutically acceptable carrier, for example an aqueous carrier.

The present invention additionally provides a formulation comprising the radiolabelled molecule of the invention, or a salt or solvate thereof. Formulations of the radiolabelled molecule may include pharmaceutically acceptable excipient(s) (carriers or diluents). Examples of generally used excipients include, without limitation: saline, buffered saline, dextrose, water-for-injection, glycerol, ethanol, and combinations thereof, stabilising agents, solubilising agents and surfactants, buffers and preservatives, tonicity agents, bulking agents, and lubricating agents.

The compositions and formulations of the invention may comprise one type of radiolabelled molecule, or more than one type of radiolabelled molecule (eg wherein the radiolabelled molecules may have the same or different dysfunctional P2X₇ receptor epitope moieties).

The compositions and formulations may be suitable for use in the methods and uses for detecting an immune cell expressing a receptor comprising an antigen recognition domain for binding a dysfunctional P2X₇ receptor and a signalling domain as described herein.

The radiolabelled molecule, which may be in a composition or formulation of the invention, may be administered to a subject using modes and techniques known to the skilled artisan. Exemplary modes include, but are not limited to: intravenous, intraperitoneal, and intratumoural injection. Other modes include, without limitation, intradermal, subcutaneous (s.c, s.q., sub-Q, Hypo), intramuscular (i.m.), intra-arterial, intramedullary, intracavital, intracardiac, intra-articular (joint), intrasynovial (joint fluid area), intracranial, intraspinal, and intrathecal (spinal fluids).

Compositions and formulations comprising the radiolabelled molecule may be administered to a subject in an amount that is effective for detecting the radiolabelled molecule, for example by a radionuclide scan. The dose may be suitably selected depending on the radionuclide present in the radiolabelled molecule. The dose may further be suitably selected depending on fluid volumes, viscosities, body weight and the like in accordance with the intended use and the particular mode of administration. A physician may ultimately determine appropriate dosages to be used.

### Kits

The present invention additionally provides a kit comprising one or more of the following:
(i) a radiolabelled molecule of the invention, or a salt or solvate thereof;
(ii) a radiolabel-precursor molecule of the invention, or a salt or solvate thereof;
(iii) a composition of the invention; or
(iv) a formulation of the invention.

In the case of a kit comprising a radiolabel-precursor compound of the invention (including a composition or a formulation comprising same), the kit may be used for preparing a radiolabelled molecule from the radiolabelled-precursor molecule, for example via the methods described herein.

The kit may be used for detecting an immune cell expressing a receptor comprising an antigen recognition domain for binding a dysfunctional P2X₇ receptor and a signalling domain in a subject who has been administered the immune cell.

Optionally a kit of the invention may further comprise an immune cell expressing a receptor comprising an antigen recognition domain for binding a dysfunctional P2X₇ receptor and a signalling domain.

Optionally a kit of the invention is packaged with instructions for use in one or more methods described herein.

### EXAMPLES

### Example 1: Preparation of Exemplary Radiolabelled Molecules

Radiolabelled E200 peptide comprising the amino acid sequence GHNYTTRNILPGLNITSTFHKTSGSGK is made by combining about 2900 g/mol of the biotinylated peptide with Ga 68 (70 g/mol).

Simple conjugation of radiolabel is via the two histidine residues in the peptide, using the method described in Mueller et al., (2016) Nature Protocols, 11: 1057-1066. Briefly, the peptide is conjugated to the chelator DOTA using standard techniques, followed by conjugated to ⁶⁸Ga.

In an alternative example, an Fc fusion protein comprising an epitope of nfP2X₇ receptor (such as having the amino acid sequence of SEQ ID NO: 145 (DetR1, monomeric; Fc attenuated; or DetR2 SEQ ID NO: 146; or dimeric Fc attenuation SEQ ID NO: 149), is conjugated to a radiolabel using a similar approach.

### Example 2: Detection of Radiolabelled Molecule for Binding nfP2X₇ Receptor Binding CAR T Cells: Imaging Study Design

A NOD.Cg-Prkdcscid Il2rgtm1Wjl/SzJ mouse model is used with an orthopic application of the breast cancer cell line MDA-MB-231 (ATCC HTB-26) into the fourth mammary fat pad at 5×10⁶ on day 0. On day 7 of the preclinical study 5E0⁶ nfP2X₇ targeted CAR T cells are injected intravenously into the tail vein.

In order to detect the presence of the CAR T cells in the mice, the mice are administered a radiolabelled molecule (ie a peptide or fusion protein) made in Example 1 via tail vein injection.

Mice are allocated into one of the following groups (n=3 per group) each representing a different time period (T) between administration of the radiolabelled molecule and detection using positron emission tomography:
control (i.e., no radiolabelled molecule administered);
T=0
T=5 min
T=10 min
T=15 min
T=20 min
T=60 min
T=120 min
T=240 min

Distribution of radiolabelled peptide or radiolabelled Fc fusion protein is assessed at each time point, via positron emission tomography (PET) scanning to detect positron emission of gallium 68.

A whole-body static PET image is acquired followed by a whole-body CT scan for anatomical reference.

A high positron emission is detected at the site of the tumour, indicating enrichment of anti-dysfunctional P2X₇ receptor CAR T cells and localisation of the cells at the tumour site.

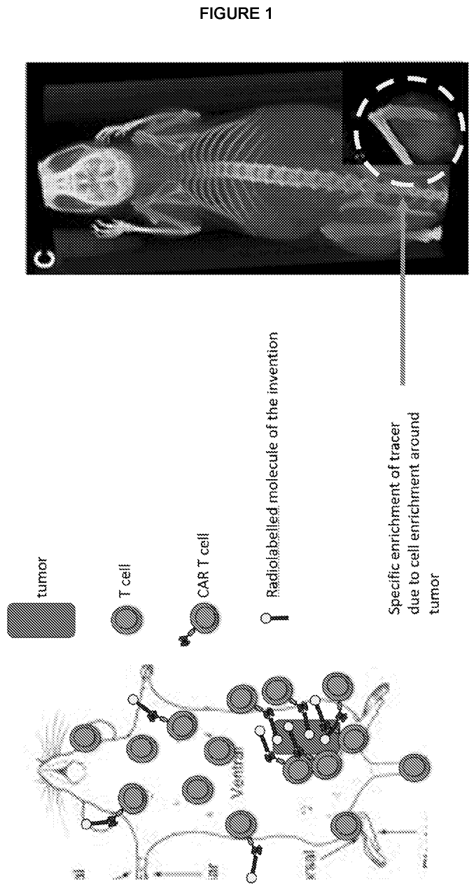

A schematic of the approach is shown in FIG. 1.

### Example 3: Demonstration of Ability of Detection Reagent to Bind to CAR T Cells In Vivo

Mice bearing pancreatic cancer cell line derived AsPC-1 tumours (administered at a dose of 0.8E06 on day −7) were infused with E200 targeted CAR T cells (ie CAR T cells capable of binding to E200 epitope as herein described) on day 0, injected intravenously into the tail vein.

Mice were subsequently injected intraperitoneally with 50 μg of a monomeric E200-Fc fusion protein (eg comprising the amino acid sequence of SEQ ID NO: 145) and comprising a C-terminal His-tag. One hour was allowed to elapse in order to allow the fusion protein to bind to CAR T cells in vivo.

Three samples of whole blood and bone marrow were taken from the mice and subjected to flow cytometry using an anti-His tag-FITC antibody (VioGreen).

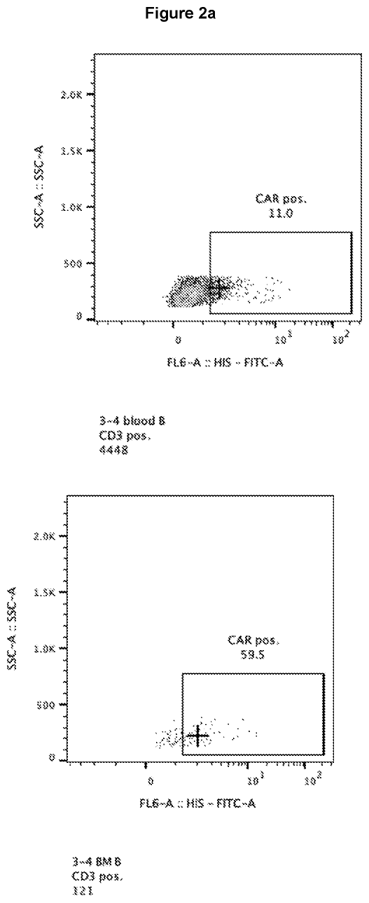

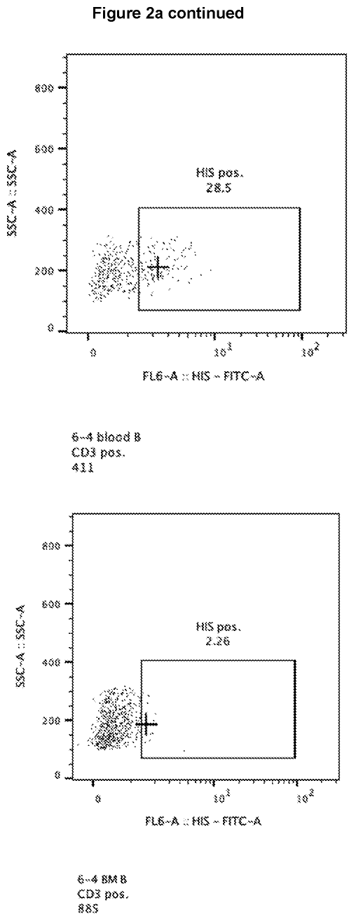

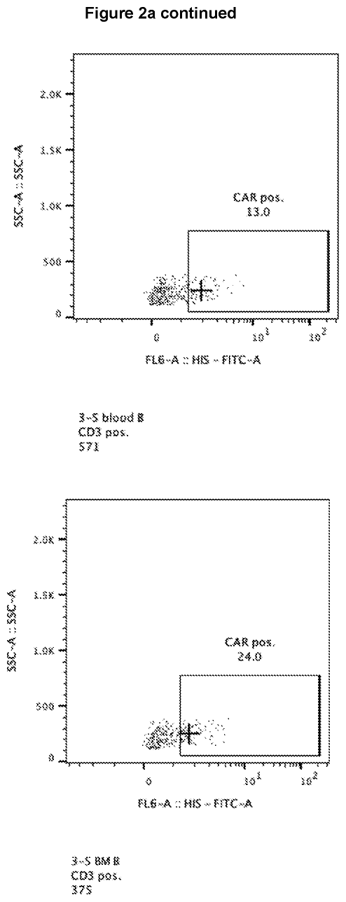

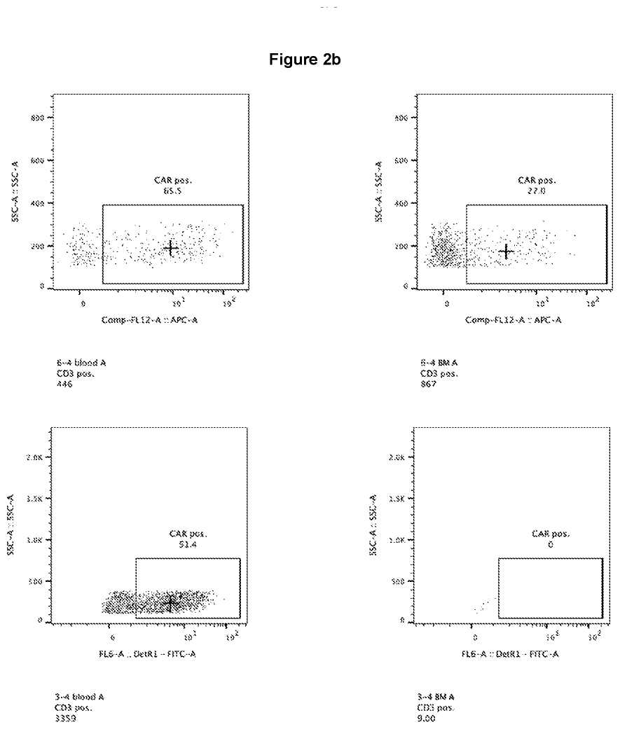

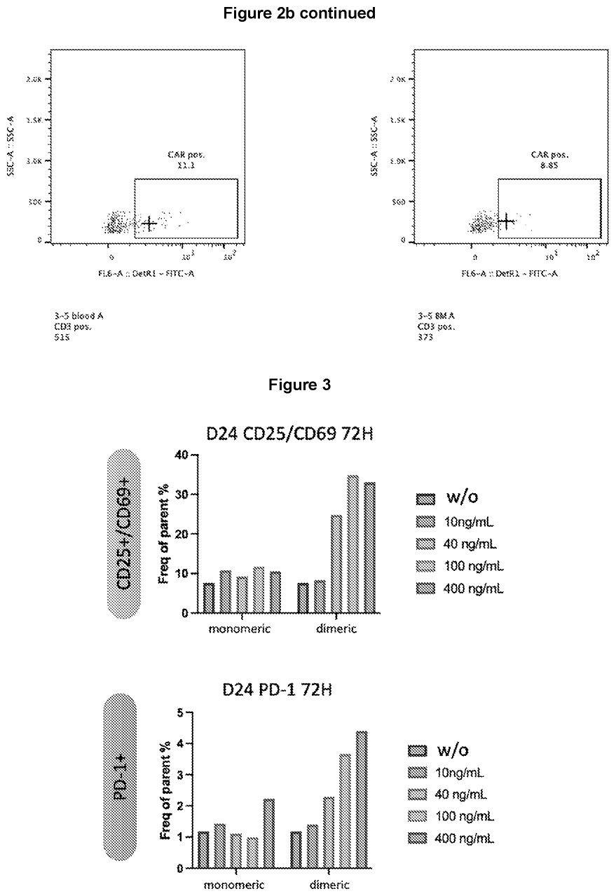

Briefly, for the results shown in FIG. 2a (using a His-tagged monomeric fusion protein of the invention, eg having the amino acid sequence of SEQ ID NO: 158):
A) blood was lysed and stained with anti-HIS-tag antibody conjugated to FITC to detect the monomeric fusion protein reagent bound to CAR-expressing cells.
B) bone marrow was isolated from the femur and stained with an anti-HIS antibody conjugated to FITC to detect monomeric fusion protein bound to CAR-expressing cells.

The anti HIS-Tag antibody was used according to manufacturer's instructions. Data was acquired on a MACSQuant16 Flow cytometer, Miltenyi.

For the results shown in FIG. 2b (using a LCLC-biotin conjugated monomeric fusion protein of the invention, eg having the amino acid sequence of SEQ ID NO: 158):
A) blood was lysed and incubated with a LCLC biotin conjugated monomeric fusion protein and then stained with a commercial anti-biotin antibody in FITC (VioGreen) to detect the monomeric detection reagent bound to the CAR expressing cells.
B) Bone marrow was isolated then incubated with a LCLC biotin conjugated monomeric fusion protein and then stained with a commercial anti-biotin antibody conjugated with FITC (VioGreen) or APC to detect the monomeric detection reagent bound to the CAR expressing cells.

Anti-biotin antibody was used according to manufacturer's instructions. Data was acquired on a MACSQuant16 Flow cytometer, Miltenyi.

The results, shown in FIG. 2a demonstrate that in vivo bound monomeric molecule is capable of binding to CAR-expressing cells. The molecules were detected via anti-HIS antibody ex vivo by flow cytometry.

The results shown in FIG. 2b show that PBMC staining from mouse blood and bone marrow via ex vivo incubation of biotinylated monomeric molecule was detectable via anti-biotin antibody to identify the CAR expressing cell subset.

Overall the results show that CAR T cells can be bound in vivo using a monomeric fusion protein as described herein.

In parallel, a series of in vitro experiment were performed wherein briefly, Jurkat cells and/or primary CD4+ T cells and CD8+ T cells (mixed at 1:1 ratio after enrichment) from a healthy volunteer donor were stably transduced by lentivirus (3rd gen LV system) to express an anti-nfP2X₇ chimeric antigen receptor (CAR), wherein the CAR comprised an antigen binding domain for binding to the E200 epitope of the P2X₇ receptor.

CAR T cells were contacted using either a monomeric fusion protein or a dimeric fusion protein, each comprising a peptide moiety capable of being bound by the CAR or protein. The fusion proteins used in this experiment comprise the amino acid sequences of SEQ ID NOs: 158 (monomeric) and 149 (dimeric).

FIG. 3 shows the levels of CD25+/CD69+ expression (each measures of T cell activation) and the levels of PD-1 expression (a measure of T cell exhaustion), at up to 72 hours following contact with varying concentrations of the fusion proteins (10 ng/ml to 400 ng/ml).

The results show that contacting CAR T cells using a monomeric fusion protein leads to significantly less T cell activation and significantly less T cell exhaustion, in a concentration dependent manner, compared to when using a dimeric fusion protein. These results indicate that for the purposes of in vivo imaging of CAR T cells, it is preferable to use a monomeric fusion protein in order to minimise unwanted activation and exhaustion of the CAR T cells in the patient.

Similar experiments are conducted using heterodimeric asymmetric molecules as described herein (eg such that the molecules comprise dimerisation between an E200 peptide-Fc fusion protein and a non-identical Fc region of an antibody; using KIH technology). The results similarly show that contacting CAR T cells using a heterodimeric asymmetric molecule comprising a single copy of the E200 peptide sequence, leads to significantly less T cell activation and significantly less T cell exhaustion, in a concentration dependent manner, compared to when using a dimeric fusion protein that comprises two copies of the E200 peptide (eg wherein the dimer is a homodimer of E200-Fc fusion proteins). These results indicate that for the purposes of in vivo imaging of CAR T cells, it is preferable to use an asymmetric heterodimer molecule or monomeric fusion protein (ie comprising a single E200 peptide sequence) in order to minimise unwanted activation and exhaustion of the CAR T cells in the patient.

1. A radiolabelled molecule comprising:
(i) a dysfunctional P2X₇ receptor epitope moiety that is recognised or capable of being bound by an antigen binding domain of a receptor expressed on an immune cell, wherein the receptor is for binding a dysfunctional P2X₇ receptor and comprises a signalling domain; and
(ii) a radionuclide that is directly or indirectly linked to the epitope moiety,
or a salt or solvate thereof.

2. The radiolabelled molecule according to claim 1, wherein the radionuclide is a positron-emitting radioisotope or a gamma-emitting isotope.

3. The radiolabelled molecule according to claims 1 or 2, wherein the radionuclide is selected from ¹¹C, ¹⁸F, ⁴⁴Sc, ⁶²Cu, ⁶⁴Cu, ⁶⁸Ga, ⁸⁶Y, ⁸⁹Zr, ⁹⁰Nb, ⁹⁹mTc, ¹¹¹In, ¹²⁴I, ¹²⁵I, ¹³¹I, ¹⁷⁷Lu and ²¹³Bi.

4. The radiolabelled molecule according to any one of claims 1 to 3, wherein the radionuclide is directly linked to an amino acid side chain of the epitope moiety.

5. The radiolabelled molecule according to any one of claims 1 to 4, wherein the molecule comprises a radiolabelled moiety conjugated to the epitope moiety.

6. The radiolabelled molecule according to claim 5, wherein the radiolabelled moiety comprises a covalently bound radionuclide

7. The radiolabelled molecule according to claim 5, wherein the radiolabelled moiety comprises a chelator moiety that is capable of chelating a radionuclide, wherein a radionuclide is complexed with the chelator moiety.

8. The radiolabelled compound according to claim 7, wherein the chelator moiety is selected from TMT, DOTA, TCMC, DO3A, CB-DO2A, NOTA, NETA, diamsar, DTPA, CHX-A″-DTPA, TETA, HBED, 5HBED, HYBIC, DFO, DFOsq and HOPO.

9. The radiolabelled compound according to any one of claims 5 to 8, wherein the radiolabelled moiety is conjugated to a further dysfunctional P2X₇ receptor epitope moiety that is recognised or capable of being bound by an antigen binding domain of a receptor expressed on an immune cell, wherein the receptor comprises is for binding a dysfunctional P2X₇ receptor and comprises a signalling domain.

10. A radiolabel-precursor molecule comprising:
(i) a dysfunctional P2X₇ receptor epitope moiety that is recognised or capable of being bound by an antigen binding domain of a receptor expressed on an immune cell, wherein the receptor is for binding a dysfunctional P2X₇ receptor and comprises a signalling domain; and
(ii) a radionuclide precursor moiety selected from:
(A) an atom or functionality capable of being converted to a radionuclide;
(B) a reactive functionality capable of conjugating to a radiolabelled prosthetic group; or
(C) a chelator moiety capable of chelating a radionuclide,
or a salt or solvate thereof.

11. The radiolabel-precursor molecule according to claim 10, wherein the radionuclide precursor moiety is conjugated to the epitope moiety.

12. The radiolabel-precursor molecule according to claim 11, wherein the radiolabel-precursor moiety comprises a chelator moiety selected from TMT, DOTA, TCMC, DO3A, CB-DO2A, NOTA, NETA, diamsar, DTPA, CHX-A″-DTPA, TETA, HBED, 5HBED, HYBIC, DFO, DFOsq and HOPO.

13. The radiolabel-precursor molecule according to claim 11 or claim 12, wherein the radiolabel-precursor moiety is further conjugated to a further dysfunctional P2X₇ receptor epitope moiety that is recognised or capable of being bound by a receptor expressed on an immune cell, wherein the receptor comprises an antigen recognition domain for binding a dysfunctional P2X₇ receptor and a signalling domain.

14. The radiolabelled molecule according to any one of claims 1 to 9, or the radio-label precursor molecule according to any one of claims 10 to 13, wherein the epitope moiety comprises or consists of an amino acid sequence selected from any one of SEQ ID NOs: 2 to 69 or 122.

15. The radiolabelled molecule according to any one of claims 1 to 9, or the radio-label precursor molecule according to any one of claims 10 to 13, wherein the epitope moiety comprises or consists of at least the amino acid sequence as set forth in SEQ ID NOs: 7 or 14.

16. The radiolabelled molecule according to any one of claims 1 to 9, or the radio-label precursor molecule according to any one of claims 10 to 13, wherein the molecule is in the form of a fusion protein.

17. A fusion protein comprising the radiolabelled molecule according to any one of claims 1 to 9, or the radio-label precursor molecule according to any one of claims 10 to 13.

18. The fusion protein of claim 17, wherein the fusion protein comprises a dysfunctional P2X₇ receptor epitope moiety and an Fc region of an antibody.

19. The fusion protein of claim 18, wherein the Fc region of an antibody comprises at least the CH₂ and CH₃ domains of an immunoglobulin.

20. The fusion protein of claims 18 or 19, wherein the Fc region comprises one or more amino acid substitutions for preventing homodimerisation of the Fc region.

21. The fusion protein of claim 20, wherein the amino acid substitutions comprise one or more substitutions of cysteine residues.

22. The fusion protein of any one of claims 18 to 21, wherein the Fc region further comprises one or more amino acid substitutions for reducing binding to any of FcγRI, FcγRII and FcγRIII, and/or one or more substitutions for reducing binding to FcRn.

23. The fusion protein of any one of claims 18 to 22, wherein the protein comprises or consists of the amino acid sequence as set forth in any of SEQ ID NOs: 145 to 158, 160 and 161.

24. A method of preparing a radiolabelled molecule, comprising:
providing a radiolabel-precursor molecule as defined in any one of claims 10 to 16 or fusion protein of any one of claims 17 to 23; and
reacting the radiolabel-precursor molecule under conditions suitable for:
(i) converting the atom or functionality of (A) to a radionuclide;
(ii) conjugating the reactive functionality of (B) to a radiolabelled prosthetic group; or
(iii) chelating the chelator moiety of (C) to a radionuclide.

25. A radiolabelled molecule obtained according to the method of claim 24.

26. A radiolabelled, asymmetric heterodimeric molecule comprising a fusion protein of any one of claims 18 to 23.

27. Use of the radiolabelled molecule according to any one of claims 1 to 9, or 25 or 26, for detecting an immune cell, or population of immune cells, expressing a receptor comprising an antigen recognition domain for binding a dysfunctional P2X₇ receptor and a signalling domain.

28. A method of detecting an immune cell, or population of immune cells, expressing a receptor comprising an antigen recognition domain for binding a dysfunctional P2X₇ receptor and a signalling domain in a subject, comprising:
administering the radiolabelled molecule according to any one of claims 1 to 9 or 25 or 26, to a subject who has been administered an immune cell expressing a receptor comprising an antigen recognition domain for binding a dysfunctional P2X₇ receptor and a signalling domain; and
detecting the radiolabelled molecule in the subject, wherein presence of the radiolabelled molecule indicates the presence of the immune cell.

29. The method according to claim 28, wherein the radiolabelled molecule is detected by performing a radionuclide scan.

30. A composition comprising the radiolabelled molecule or the radio-label precursor molecule according to any one of claims 1 to 16, the radiolabelled molecule of claim 25 or 26, or the fusion protein of any one of claims 17 to 23, or a salt or solvate thereof.

31. A formulation comprising the radiolabelled molecule or the radio-label precursor molecule according to any one of claims 1 to 16, the radiolabelled molecule of claim 25 or 26, or the fusion protein of any one of claims 17 to 23, or a salt or solvate thereof.

32. A kit comprising one or more of the following:
(i) the radiolabelled molecule according to any one of claims 1 to 9, or 25 or 26 or a salt or solvate thereof;
(ii) the radiolabel-precursor molecule according to any one of claims 10 to 15, or a salt or solvate thereof;
(iii) the fusion protein according to any one of claims 17 to 23;
(iv) the composition according to claim 30; or
(v) the formulation according to claim 31.


SEQUENCE LISTING (162 sequences)

SEQ ID NO: 1 - protein, 595 aa, synthetic construct
MPACCSCSDVFQYETNKVTRIQSMNYGTIKWFFHVIIFSYVCFALVSDKLYQRKEPVISSVHTKVKGIAEVKEEIVENGVKKLVHSVFDTADYTFPLQGNSFFVMTNFLKTEGQEQRLCPEYPTRRTLCSSDRGCKKGWMDPQSKGIQTGRCVVYEGNQKTCEVSAWCPIEAVEEAPRPALLNSAENFTVLIKNNIDFPGHNYTTRNILPGLNITCTFHKTQNPQCPIFRLGDIFRETGDNFSDVAIQGGIMGIEIYWDCNLDRWFHHCRPKYSFRRLDDKTTNVSLYPGYNFRYAKYYKENNVEKRTLIKVFGIRFDILVFGTGGKFDIIQLVVYIGSTLSYFGLAAVFIDFLIDTYSSNCCRSHIYPWCKCCQPCVVNEYYYRKKCESIVEPKPTLKYVSFVDESHIRMVNQQLLGRSLQDVKGQEVPRPAMDFTDLSRLPLALHDTPPIPGQPEEIQLLRKEATPRSRDSPVWCQCGSCLPSQLPESHRCLEELCCRKKPGACITTSELFRKLVLSRHVLQFLLLYQEPLLALDVDSTNSRLRHCAYRCYATWRFGSQDMADFAILPSCCRWRIRKEFPKSEGQYSGFKSPY

SEQ ID NO: 2 - protein, 17 aa, synthetic construct
GHNYTTRNILPGLNITC

SEQ ID NO: 3 - protein, 16 aa, synthetic construct
GHNYTTRNILPGLNIT

SEQ ID NO: 4 - protein, 15 aa, synthetic construct
KYYKENNVEKRTLIK

SEQ ID NO: 5 - protein, 17 aa, synthetic construct
KYYKENNVEKRTLIKVF

SEQ ID NO: 6 - protein, 25 aa, synthetic construct
GHNYTTRNILPGAGAKYYKENNVEK

SEQ ID NO: 7 - protein, 17 aa, synthetic construct
GHNYTTRNILPGLNITS

SEQ ID NO: 8 - protein, 21 aa, synthetic construct
GHNYTTRNILPGLNITSTFHK

SEQ ID NO: 9 - protein, 22 aa, synthetic construct
GHNYTTRNILPGLNITSTFHKT

SEQ ID NO: 10 - protein, 23 aa, synthetic construct
GHNYTTRNILPGLNITSTFHKTC

SEQ ID NO: 11 - protein, 27 aa, synthetic construct
GHNYTTRNILPGLNITSTFHKTSGSGK

SEQ ID NO: 12 - protein, 23 aa, synthetic construct
GHNYTTRNILPGLNITSTFHKTS

SEQ ID NO: 13 - protein, 16 aa, synthetic construct
DFPGHNYTTRNILPGC

SEQ ID NO: 14 - protein, 11 aa, synthetic construct
NYTTRNILPGL

SEQ ID NO: 15 - protein, 22 aa, synthetic construct
GHNYTTRNILPGLNITSGGGGS

SEQ ID NO: 16 - protein, 27 aa, synthetic construct
GHNYTTRNILPGLNITSGGGGSGGGGS

SEQ ID NO: 17 - protein, 32 aa, synthetic construct
GHNYTTRNILPGLNITSGGGGSGGGGSGGGGS

SEQ ID NO: 18 - protein, 24 aa, synthetic construct
GHNYTTRNILPGLNITSTFHKTGS

SEQ ID NO: 19 - protein, 22 aa, synthetic construct
GHNYTTRNILPGLNITSTFHGS

SEQ ID NO: 20 - protein, 19 aa, synthetic construct
GHNYTTRNILPGLNITSGS

SEQ ID NO: 21 - protein, 22 aa, synthetic construct
DFPGHNYTTRNILPGLNITSGS

SEQ ID NO: 22 - protein, 25 aa, synthetic construct
DFPGHNYTTRNILPGLNITSGGGGS

SEQ ID NO: 23 - protein, 30 aa, synthetic construct
DFPGHNYTTRNILPGLNITSGGGGSGGGGS

SEQ ID NO: 24 - protein, 35 aa, synthetic construct
DFPGHNYTTRNILPGLNITSGGGGSGGGGSGGGGS

SEQ ID NO: 25 - protein, 30 aa, synthetic construct
DFPGHNYTTRNILPGLNITSTFHKTSGSGK

SEQ ID NO: 26 - protein, 32 aa, synthetic construct
DFPGHNYTTRNILPGLNITSTFHKTSGSGKGS

SEQ ID NO: 27 - protein, 35 aa, synthetic construct
DFPGHNYTTRNILPGLNITSTFHKTSGSGKGGGGS

SEQ ID NO: 28 - protein, 28 aa, synthetic construct
DFPGHNYTTRNILPGLNITSTFHGGGGS

SEQ ID NO: 29 - protein, 25 aa, synthetic construct
GHNYTTRNILPGLNITSTFHGGGGS

SEQ ID NO: 30 - protein, 30 aa, synthetic construct
DFPGHNYTTRNILPGLNITSTFHKTGGGGS

SEQ ID NO: 31 - protein, 27 aa, synthetic construct
GHNYTTRNILPGLNITSTFHKTGGGGS

SEQ ID NO: 32 - protein, 27 aa, synthetic construct
GHNYTTRNILPGLNITSEPKSSDKTHT

SEQ ID NO: 33 - protein, 29 aa, synthetic construct
GHNYTTRNILPGLNITSGSEPKSSDKTHT

SEQ ID NO: 34 - protein, 32 aa, synthetic construct
GHNYTTRNILPGLNITSGGGGSEPKSSDKTHT

SEQ ID NO: 35 - protein, 37 aa, synthetic construct
GHNYTTRNILPGLNITSTFHKTSGSGKEPKSSDKTHT

SEQ ID NO: 36 - protein, 39 aa, synthetic construct
GHNYTTRNILPGLNITSTFHKTSGSGKGSEPKSSDKTHT

SEQ ID NO: 37 - protein, 42 aa, synthetic construct
GHNYTTRNILPGLNITSTFHKTSGSGKGGGGSEPKSSDKTHT

SEQ ID NO: 38 - protein, 29 aa, synthetic construct
GHNYTTRNILPGLNITSEPKSSDKTHTGS

SEQ ID NO: 39 - protein, 31 aa, synthetic construct
GHNYTTRNILPGLNITSGSEPKSSDKTHTGS

SEQ ID NO: 40 - protein, 34 aa, synthetic construct
GHNYTTRNILPGLNITSGGGGSEPKSSDKTHTGS

SEQ ID NO: 41 - protein, 39 aa, synthetic construct
GHNYTTRNILPGLNITSTFHKTSGSGKEPKSSDKTHTGS

SEQ ID NO: 42 - protein, 41 aa, synthetic construct
GHNYTTRNILPGLNITSTFHKTSGSGKGSEPKSSDKTHTGS

SEQ ID NO: 43 - protein, 44 aa, synthetic construct
GHNYTTRNILPGLNITSTFHKTSGSGKGGGGSEPKSSDKTHTGS

SEQ ID NO: 44 - protein, 32 aa, synthetic construct
GHNYTTRNILPGLNITSEPKSSDKTHTGGGGS

SEQ ID NO: 45 - protein, 34 aa, synthetic construct
GHNYTTRNILPGLNITSGSEPKSSDKTHTGGGGS

SEQ ID NO: 46 - protein, 37 aa, synthetic construct
GHNYTTRNILPGLNITSGGGGSEPKSSDKTHTGGGGS

SEQ ID NO: 47 - protein, 42 aa, synthetic construct
GHNYTTRNILPGLNITSTFHKTSGSGKEPKSSDKTHTGGGGS

SEQ ID NO: 48 - protein, 44 aa, synthetic construct
GHNYTTRNILPGLNITSTFHKTSGSGKGSEPKSSDKTHTGGGGS

SEQ ID NO: 49 - protein, 47 aa, synthetic construct
GHNYTTRNILPGLNITSTFHKTSGSGKGGGGSEPKSSDKTHTGGGGS

SEQ ID NO: 50 - protein, 30 aa, synthetic construct
DFPGHNYTTRNILPGLNITSEPKSSDKTHT

SEQ ID NO: 51 - protein, 32 aa, synthetic construct
DFPGHNYTTRNILPGLNITSGSEPKSSDKTHT

SEQ ID NO: 52 - protein, 35 aa, synthetic construct
DFPGHNYTTRNILPGLNITSGGGGSEPKSSDKTHT

SEQ ID NO: 53 - protein, 40 aa, synthetic construct
DFPGHNYTTRNILPGLNITSTFHKTSGSGKEPKSSDKTHT

SEQ ID NO: 54 - protein, 42 aa, synthetic construct
DFPGHNYTTRNILPGLNITSTFHKTSGSGKGSEPKSSDKTHT

SEQ ID NO: 55 - protein, 45 aa, synthetic construct
DFPGHNYTTRNILPGLNITSTFHKTSGSGKGGGGSEPKSSDKTHT

SEQ ID NO: 56 - protein, 32 aa, synthetic construct
DFPGHNYTTRNILPGLNITSEPKSSDKTHTGS

SEQ ID NO: 57 - protein, 34 aa, synthetic construct
DFPGHNYTTRNILPGLNITSGSEPKSSDKTHTGS

SEQ ID NO: 58 - protein, 37 aa, synthetic construct
DFPGHNYTTRNILPGLNITSGGGGSEPKSSDKTHTGS

SEQ ID NO: 59 - protein, 42 aa, synthetic construct
DFPGHNYTTRNILPGLNITSTFHKTSGSGKEPKSSDKTHTGS

SEQ ID NO: 60 - protein, 44 aa, synthetic construct
DFPGHNYTTRNILPGLNITSTFHKTSGSGKGSEPKSSDKTHTGS

SEQ ID NO: 61 - protein, 47 aa, synthetic construct
DFPGHNYTTRNILPGLNITSTFHKTSGSGKGGGGSEPKSSDKTHTGS

SEQ ID NO: 62 - protein, 35 aa, synthetic construct
DFPGHNYTTRNILPGLNITSEPKSSDKTHTGGGGS

SEQ ID NO: 63 - protein, 37 aa, synthetic construct
DFPGHNYTTRNILPGLNITSGSEPKSSDKTHTGGGGS

SEQ ID NO: 64 - protein, 40 aa, synthetic construct
DFPGHNYTTRNILPGLNITSGGGGSEPKSSDKTHTGGGGS

SEQ ID NO: 65 - protein, 45 aa, synthetic construct
DFPGHNYTTRNILPGLNITSTFHKTSGSGKEPKSSDKTHTGGGGS

SEQ ID NO: 66 - protein, 47 aa, synthetic construct
DFPGHNYTTRNILPGLNITSTFHKTSGSGKGSEPKSSDKTHTGGGGS

SEQ ID NO: 67 - protein, 50 aa, synthetic construct
DFPGHNYTTRNILPGLNITSTFHKTSGSGKGGGGSEPKSSDKTHTGGGGS

SEQ ID NO: 68 - protein, 40 aa, synthetic construct
DKTHTSPPSPAPELLGGGGSDFPGHNYTTRNILPGLNITS

SEQ ID NO: 69 - protein, 50 aa, synthetic construct
GGGGSEPKSSDKTHTSPPSPAPELLGGGGSDFPGHNYTTRNILPGLNITS

SEQ ID NO: 70 - protein, 5 aa, synthetic construct
GGGGS

SEQ ID NO: 71 - protein, 10 aa, synthetic construct
EPKSSDKTHT

SEQ ID NO: 72 - protein, 22 aa, synthetic construct
GSEPKSSDKTHTSPPSPAPELL

SEQ ID NO: 73 - protein, 30 aa, synthetic construct
GGGGSEPKSSDKTHTSPPSPAPELLGGGGS

SEQ ID NO: 74 - protein, 12 aa, synthetic construct
EPKSSDKTHTGS

SEQ ID NO: 75 - protein, 15 aa, synthetic construct
EPKSSDKTHTGGGGS

SEQ ID NO: 76 - protein, 15 aa, synthetic construct
EPKSSDKTHTSPPSP

SEQ ID NO: 77 - protein, 13 aa, synthetic construct
EPKSSDKTHTSPP

SEQ ID NO: 78 - protein, 14 aa, synthetic construct
EPKSSDKTHTSPPP

SEQ ID NO: 79 - protein, 10 aa, synthetic construct
EPKSSDKTHT

SEQ ID NO: 80 - protein, 10 aa, synthetic construct
DKTHTSPPSP

SEQ ID NO: 81 - protein, 8 aa, synthetic construct
DKTHTSPP

SEQ ID NO: 82 - protein, 12 aa, synthetic construct
EPKSDKTHTPPP

SEQ ID NO: 83 - protein, 14 aa, synthetic construct
EPKSDKTHTSPPSP

SEQ ID NO: 84 - protein, 15 aa, synthetic construct
EPKSGDKTHTGPPGP

SEQ ID NO: 85 - protein, 13 aa, synthetic construct
EPKSGDKTHTGPP

SEQ ID NO: 86 - protein, 14 aa, synthetic construct
EPKSGDKTHTGPPP

SEQ ID NO: 87 - protein, 10 aa, synthetic construct
EPKSGDKTHT

SEQ ID NO: 88 - protein, 10 aa, synthetic construct
DKTHTGPPGP

SEQ ID NO: 89 - protein, 8 aa, synthetic construct
DKTHTGPP

SEQ ID NO: 90 - protein, 14 aa, synthetic construct
EPKSDKTHTGPPGP

SEQ ID NO: 91 - protein, 15 aa, synthetic construct
EPKSGDKTHTSPPSP

SEQ ID NO: 92 - protein, 15 aa, synthetic construct
EPKSGDKTHTGPPSP

SEQ ID NO: 93 - protein, 15 aa, synthetic construct
EPKSSDKTHTGPPGP

SEQ ID NO: 94 - protein, 15 aa, synthetic construct
EPKSSDKTHTGPPSP

SEQ ID NO: 95 - protein, 13 aa, synthetic construct
EPKSSDKTHTGPP

SEQ ID NO: 96 - protein, 13 aa, synthetic construct
EPKSGDKTHTSPP

SEQ ID NO: 97 - protein, 15 aa, synthetic construct
EPKSTDKTHTTPPTP

SEQ ID NO: 98 - protein, 13 aa, synthetic construct
EPKSTDKTHTTPP

SEQ ID NO: 99 - protein, 14 aa, synthetic construct
EPKSTDKTHTTPPP

SEQ ID NO: 100 - protein, 10 aa, synthetic construct
EPKSTDKTHT

SEQ ID NO: 101 - protein, 10 aa, synthetic construct
DKTHTTPPTP

SEQ ID NO: 102 - protein, 8 aa, synthetic construct
DKTHTTPP

SEQ ID NO: 103 - protein, 14 aa, synthetic construct
EPKSDKTHTTPPTP

SEQ ID NO: 104 - protein, 15 aa, synthetic construct
EPKSSDKTHTTPPTP

SEQ ID NO: 105 - protein, 15 aa, synthetic construct
EPKSSDKTHTSPPTP

SEQ ID NO: 106 - protein, 15 aa, synthetic construct
EPKSADKTHTLPPMP

SEQ ID NO: 107 - protein, 15 aa, synthetic construct
EPKSVDKTHTLPPIP

SEQ ID NO: 108 - protein, 15 aa, synthetic construct
EPKSLDKTHTAPPAP

SEQ ID NO: 109 - protein, 13 aa, synthetic construct
EPKSVDKTHTAPP

SEQ ID NO: 110 - protein, 13 aa, synthetic construct
EPKSIDKTHTLPP

SEQ ID NO: 111 - protein, 10 aa, synthetic construct
DKTHTAPPLP

SEQ ID NO: 112 - protein, 10 aa, synthetic construct
DKTHTVPPLP

SEQ ID NO: 113 - protein, 12 aa, synthetic construct
EPKSSDKTHTSP

SEQ ID NO: 114 - protein, 15 aa, synthetic construct
EPKSCDKTHTCPPCP

SEQ ID NO: 115 - protein, 12 aa, synthetic construct
ERKCCVECPPCP

SEQ ID NO: 116 - protein, 12 aa, synthetic construct
ERKXXVEXPPXP

SEQ ID NO: 117 - protein, 10 aa, synthetic construct
ERKXXVEXPP

SEQ ID NO: 118 - protein, 7 aa, synthetic construct
VEXPPXP

SEQ ID NO: 119 - protein, 17 aa, synthetic construct
ELKTPLGDTTHTCPRCP

SEQ ID NO: 120 - protein, 17 aa, synthetic construct
ELKTPLGDTTHTXPRXP

SEQ ID NO: 121 - protein, 15 aa, synthetic construct
ELKTPLGDTTHTXPR

SEQ ID NO: 122 - protein, 25 aa, synthetic construct
DFPGHNYTTRNILPGLNITSTFHKT

SEQ ID NO: 123 - protein, 12 aa, synthetic construct
ELKTPLGDTTHT

SEQ ID NO: 124 - protein, 15 aa, synthetic construct
EPKSCDTPPPCPRCP

SEQ ID NO: 125 - protein, 15 aa, synthetic construct
EPKSXDTPPPXPRXP

SEQ ID NO: 126 - protein, 13 aa, synthetic construct
EPKSXDTPPPXPR

SEQ ID NO: 127 - protein, 10 aa, synthetic construct
EPKSXDTPPP

SEQ ID NO: 128 - protein, 10 aa, synthetic construct
DTPPPXPRXP

SEQ ID NO: 129 - protein, 12 aa, synthetic construct
ESKYGPPCPSCP

SEQ ID NO: 130 - protein, 12 aa, synthetic construct
EXKYGPPCPXCP

SEQ ID NO: 131 - protein, 9 aa, synthetic construct
EXKYGPPCP

SEQ ID NO: 132 - protein, 10 aa, synthetic construct
KYGPPCPXCP

SEQ ID NO: 133 - protein, 12 aa, synthetic construct
EPKSCDKTHTCP

SEQ ID NO: 134 - protein, 12 aa, synthetic construct
EPKSCDTPPPCP

SEQ ID NO: 135 - protein, 12 aa, synthetic construct
ESKYGPPSPSSP

SEQ ID NO: 136 - protein, 12 aa, synthetic construct
ERKSSVESPPSP

SEQ ID NO: 137 - protein, 12 aa, synthetic construct
EPKSSDTPPPSP

SEQ ID NO: 138 - protein, 5 aa, synthetic construct
TFHKT

SEQ ID NO: 139 - skipped

SEQ ID NO: 140 - protein, 20 aa, synthetic construct
DFPGHNYTTRNILPGLNITS

SEQ ID NO: 141 - protein, 17 aa, synthetic construct
EPKSCDKTHTSPPSPAP

SEQ ID NO: 142 - protein, 17 aa, synthetic construct
EPKSSDKTHTSPPSPAP

SEQ ID NO: 143 - protein, 5 aa, synthetic construct
GGGGS

SEQ ID NO: 144 - protein, 11 aa, synthetic construct
LEVLFQGPVRR

SEQ ID NO: 145 - protein, 266 aa, synthetic construct
DFPGHNYTTRNILPGLNITSTFHKTSGSGKGGGGSEPKSCDKTHTSPPSPAPPVAGPSVFLFPPKPKDTLMISRTPEVTCVVVGVSHEDPEVKFNWYVDGVEVHNAKTKPREEQYQSTYRVVSVLTVLHQDWLNGKEYKCKVSNKQLPSPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - protein, 263 aa, synthetic construct
GHNYTTRNILPGLNITSTFHKTSGSGKGGGGSEPKSCDKTHTSPPSPAPPVAGPSVFLFPPKPKDTLMISRTPEVTCVVVGVSHEDPEVKFNWYVDGVEVHNAKTKPREEQYQSTYRVVSVLTVLHQDWLNGKEYKCKVSNKQLPSPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 267 aa, synthetic construct
DFPGHNYTTRNILPGLNITSTFHKTSGSGKGGGGSEPKSSDKTHTSPPSPAPEAARGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 264 aa, synthetic construct
GHNYTTRNILPGLNITSTFHKTSGSGKGGGGSEPKSSDKTHTSPPSPAPEAARGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 266 aa, synthetic construct
DFPGHNYTTRNILPGLNITSTFHKTSGSGKGGGGSEPKSCDKTHTCPPCPAPPVAGPSVFLFPPKPKDTLMISRTPEVTCVVVGVSHEDPEVKFNWYVDGVEVHNAKTKPREEQYQSTYRVVSVLTVLHQDWLNGKEYKCKVSNKQLPSPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 263 aa, synthetic construct
GHNYTTRNILPGLNITSTFHKTSGSGKGGGGSEPKSCDKTHTCPPCPAPPVAGPSVFLFPPKPKDTLMISRTPEVTCVVVGVSHEDPEVKFNWYVDGVEVHNAKTKPREEQYQSTYRVVSVLTVLHQDWLNGKEYKCKVSNKQLPSPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 267 aa, synthetic construct
DFPGHNYTTRNILPGLNITSTFHKTSGSGKGGGGSEPKSSDKTHTCPPCPAPEAARGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 264 aa, synthetic construct
GHNYTTRNILPGLNITSTFHKTSGSGKGGGGSEPKSSDKTHTCPPCPAPEAARGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 277 aa, synthetic construct
DFPGHNYTTRNILPGLNITSTFHKTSGSGKGGGGSLEVLFQGPVRREPKSSDKTHTSPPSPAPPVAGPSVFLFPPKPKDTLMISRTPEVTCVVVGVSHEDPEVKFNWYVDGVEVHNAKTKPREEQYQSTYRVVSVLTVLHQDWLNGKEYKCKVSNKQLPSPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - protein, 277 aa, synthetic construct
DFPGHNYTTRNILPGLNITSTFHKTSGSGKGGGGSLEVLFQGPVRREPKSSDKTHTCPPCPAPPVAGPSVFLFPPKPKDTLMISRTPEVTCVVVGVSHEDPEVKFNWYVDGVEVHNAKTKPREEQYQSTYRVVSVLTVLHQDWLNGKEYKCKVSNKQLPSPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 267 aa, synthetic construct
DFPGHNYTTRNILPGLNITSTFHKTSGSGKGGGGSEPKSSDKTHTSPPSPAPEAARGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - protein, 267 aa, synthetic construct
DFPGHNYTTRNILPGLNITSTFHKTSGSGKGGGGSEPKSSDKTHTCPPCPAPEAARGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 266 aa, synthetic construct
DFPGHNYTTRNILPGLNITSTFHKTSGSGKGGGGSEPKSSDKTHTCPPCPAPPVAGPSVFLFPPKPKDTLMISRTPEVTCVVVGVSHEDPEVKFNWYVDGVEVHNAKTKPREEQYQSTYRVVSVLTVLHQDWLNGKEYKCKVSNKQLPSPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 267 aa, synthetic construct
DFPGHNYTTRNILPGLNITSTFHKTSGSGKGGGGSEPKSSDKTHTSPPSPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 159 - protein, 232 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 160 - protein, 267 aa, synthetic construct
DFPGHNYTTRNILPGLNITSTFHKTSGSGKGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 161 - protein, 267 aa, synthetic construct; identical to SEQ ID NO: 160

SEQ ID NO: 162 - protein, 232 aa, synthetic construct
EPKSSDKTHTSPPSPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGK